(19) United States
(12) Patent Application Publication
(10) Pub. No.: US 2025/0025309 A1
(43) Pub. Date: Jan. 23, 2025

(54) SYSTEMS AND METHODS FOR ORTHOPEDIC IMPLANTS

(71) Applicant: Carlsmed, Inc., Carlsbad, CA (US)

(72) Inventor: Niall Patrick Casey, Carlsbad, CA (US)

(21) Appl. No.: 18/907,405

(22) Filed: Oct. 4, 2024

Related U.S. Application Data
(63) Continuation-in-part of application No. 18/754,123, filed on Jun. 25, 2024.
Application No. 18/754,123 is a continuation of application No. 16/699,447, filed on Nov. 29, 2019, now Pat. No. 12,133,803.
(60) Provisional application No. 62/773,127, filed on Nov. 29, 2018.

(51) Int. Cl. A61F 2/30; A61B 34/10; G06F 30/10; G06F 111/16; G16H 20/40; G16H 30/20

(52) U.S. Cl. CPC A61F 2/30942; G06F 30/10; G16H 20/40; G16H 30/20; A61B 2034/105; A61B 2034/108; A61F 2002/30948; A61F 2002/30952; A61F 2002/30962; A61F 2002/3097; A61F 2002/30971; A61F 2002/30985; G06F 2111/16

(57) ABSTRACT

A computer-implemented method for designing a patient-specific orthopedic implant can include creating a user account associated with a patient. A patient-specific orthopedic implant can be designed based on patient data and imaging data. A healthcare provider can provide feedback for a design of the patient-specific orthopedic implant, treatment protocol, or other aspects of treatment. The patient can provide data and feedback.

### RELATED APPLICATIONS

This application is a continuation-in-part of U.S. Ser. No. 18/754,123, filed Jun. 25, 2024, which is a continuation of U.S. patent application Ser. No. 16/699,447, filed Nov. 29, 2019, which claims priority to U.S. Patent Application No. 62/773,127, filed Nov. 29, 2018, which are all herein incorporated by reference in their entireties.

### FIELD OF THE INVENTION

The field of the invention generally relates to orthopedic implants, including spinal implants, and methods for designing and producing them.

### BACKGROUND

Orthopedic implants are used to correct a variety of different maladies. Orthopedic surgery utilizing orthopedic implants may include one of a number of specialties, including: spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma.

Orthopedic surgery may include joint replacement or other replacement of bony anatomical structures. In some instances, patients may require joint replacement due to degeneration of the joint or another disruption to the functional aspects of the joint. Additional orthopedic surgeries may include replacement of bones or sections of bone that have been compromised due to tumor or trauma. In one situation, a patient may require replacement of a section of a bone whose structural integrity has been compromised by a tumor. Additionally, resection of a tumor of the bone may be required as part of a treatment protocol to stem the propagation of cancer. In other instances, trauma, such as a motor vehicle accident or fall, may cause a severe fracture or disruption of bones. In these cases, sections of bones or entire bones may require replacement as part of the treatment protocol. Treatment protocol may include resection of anatomy, reorganization of anatomy, repair of anatomy, and delivery of therapeutic implants.

Spine surgery may encompass one or more of the cervical, thoracic, lumbar spine, the sacrum, or the pelvis and may treat a deformity or degeneration of the spine, or related back pain, leg pain, or other body pain. Irregular spinal curvature may include scoliosis, lordosis, and kyphosis (hyper- or hypo-). Irregular spinal displacement may include spondylolisthesis, lateral displacement, or axial displacement. Other spinal disorders include osteoarthritis, lumbar degenerative disc disease or cervical degenerative disc disease, lumbar spinal stenosis or cervical spinal stenosis.

Spinal fusion surgery may be performed to set and hold purposeful changes imparted on the spine during surgery. Spinal fusion procedures include PLIF (posterior lumbar interbody fusion), ALIF (anterior lumbar interbody fusion), TLIF (transverse or transforaminal lumbar interbody fusion), or LLIF (lateral lumbar interbody fusion), including DLIF (direct lateral lumbar interbody fusion) or XLIF (extreme lateral lumbar interbody fusion).

One goal of interbody fusion is to grow bone between vertebra in order to seize the spatial relationships in a position that provides enough room for neural elements, including exiting nerve roots. An interbody implant device (or interbody implant, interbody cage, or fusion cage, or spine cage) is a prosthesis used in spinal fusion procedures to maintain relative position of vertebra and establish appropriate foraminal height and decompression of exiting nerves. Each patient may have individual or unique disease characteristics, but most implant solutions include implants (e.g., interbody implants) having standard sizes or shapes (stock implants).

Software is often used throughout the orthopedic implant design process. Software can be used to view relevant anatomy and create specifications for orthopedic implants. Software can also be used to create, view, and modify three-dimensional virtual models representative of implants, anatomy, and other components. Additionally, software can be used to better understand spatial relationships between relevant anatomical elements.

### BRIEF DESCRIPTION OF THE DRAWINGS

FIG. 1 shows a flowchart for designing a patient-specific orthopedic implant in accordance with one or more embodiments.

FIG. 2 shows a diagram describing information flow within a system for designing and manufacturing a patient-specific implant in accordance with one or more embodiments.

FIG. 3 shows a flow diagram describing one embodiment of a system for designing and manufacturing patient-specific implants.

FIG. 3A shows a diagram categorizing data for use in design of implants.

FIG. 3B shows a diagram showing relationships between users.

FIGS. 4 through 8 show several embodiments of views of displayable patient portal interfaces in accordance with various embodiments.

FIGS. 9 through 16 show several embodiments of views of displayable healthcare provider portal interfaces in accordance with various embodiments.

FIG. 17 illustrates a system for providing assistance for monitoring patients in accordance with one or more embodiments.

FIG. 18 shows a user device displaying a graphical user interface (GUI) in accordance with some implementations of the disclosed technology.

FIG. 19 shows a user device displaying a GUI in accordance with some implementations of the disclosed technology.

FIG. 20 illustrates a system for manufacturing patient-specific implants and/or monitoring patients in accordance with one or more embodiments.

### DETAILED DESCRIPTION

A patient-specific medical device and an efficient method of producing patient-specific orthopedic implants are described in the embodiments herein. Orthopedic implants and devices according to embodiments described herein may include spinal implants such as interbody implants, expandable implants, or fusion cages.

Orthopedic implants are typically intended to replace missing anatomy, correct pathological deformities, and/or restore anatomical relationships that have been compromised due to degeneration or aging. In some instances, implants can be used to replace anatomy that has been compromised due to fracture or trauma. In other instances, implants can be used to correct improper development. In other instances, implants can be used to restore relationships that have changed with the passing of time or advancement of a degenerative condition. Computing devices and software can be used throughout treatment, including imaging, diagnostics, planning, and/or design of corrective elements, including implants.

Software is typically used to view relevant internal anatomy, such as bones and soft tissue. When digital images (e.g., Magnetic Resonance Imaging (MRI) images, Computed Tomography (CT) scan images, or X-rays) of the patient's relevant anatomy are collected, they are displayed on computing systems that run image viewing software. The data from digital images can be saved in various formats to various media and transferred digitally through connected computers or networks of computers. Image data, as other digital data, can be sent through typical electronic networks by typical methods including email, file transfer protocol, and file sharing over servers. Software is further used to design implants, provide specifications for implants, and provide instructions for manufacture of orthopedic implants. The software can be computer-aided design (CAD) software or other software suitable for generating virtual models of anatomy, orthopedic implants, or the like.

Patient-specific implants can be designed for optimal fit in the negative space created by removal of anatomy and adjustment of the relative positions of bony anatomy. In spine surgery, surgical planning software can be used to virtually adjust the relative positions of vertebrae and define the space between the vertebrae. Modifying the spatial relationship between adjacent vertebrae within a virtual design space can provide a definition of the 3D space into which an interbody can be delivered. Software can further be used to compare the original pathological anatomy to a corrected anatomy. The optimal size and shape of patient-specific implants can prevent or reduce instances of implant failure.

One method of designing orthopedic implants includes the use of software designed to interface with patients, healthcare providers (HCP), and manufacturers. HCPs can be described as entities that provide care including, nurses, physicians, surgeons, clinicians, or others acting under the authority of the above.

One method of manufacturing orthopedic implants includes the use of additive manufacturing or three-dimensional printing (3DP). Additive manufacturing provides for manufacturing of complex components that could not otherwise be manufactured using traditional subtractive manufacturing (CNC machining, turning, milling, drilling, electron deposition manufacturing, broaching, rolling, stamping, etc.).

It is accepted that increasing engagement of the patient in medical treatment protocols increases the probability of improved outcomes. One method of increasing patient engagement is to encourage patient action prior to a surgical procedure. A patient who becomes personally engaged in pre-operative activities is more likely to be engaged in post-operative recovery activities, thereby improving the likelihood of improved outcomes.

A computing system can be configured to design patient-specific implants. The computing system can be configured to run software on multiple computing platforms and devices, such as desktop computers, laptop computers, tablet computes, hand-held computers, smartphones, or other devices.

The system can operate a series of steps arranged to collect and share information critical to the design and manufacture of orthopedic implants. In one embodiment, a manufacturer (company) receives a request from a surgeon for a patient-specific (e.g., custom, bespoke, personalized, matched, etc.) implant. The company then can send a request to the surgeon or qualified HCP soliciting information such as primary surgeon, digital imaging data (scan data), surgery date, treatment protocol, surgery location, patient pathology, location of pathology within patient, patient name, patient contact information, authority to contact patient directly, patient date of birth, and patient gender. A treatment protocol can be described by the surgeon or HCP and could include specific anatomy to remove, manipulate, or adjust and the implants to aid with surgical treatment of the patient's pathology. The collection of data outside of the digital imaging data can be described as meta data.

The company can contact the patient and request additional or missing information. The company can make direct requests of the patient to use their digital imaging data (or other relevant data) to design patient-specific implants. Company can implement data management and security measures to comply with Health Insurance Portability and Accountability Act including anonymizing patient data, removing electronic protected health information, and data encryption.

Digital imaging data can be acquired directly from the patient or from the HCP via electronic networks. Additionally, digital imaging data can be provided using electronic storage on portable media (such as flash drives or computer discs).

When the company has a complete file or nearly complete file with digital imaging data (e.g., scan data), patient data, HCP data, and case data, they can begin design of patient-specific implants. Following design of the patient-specific implants, the company can alert the HCP and/or patient and invite each to view the proposed treatment protocol.

Elements of a software solution can be referred to as portals or interfaces. Portals can be designed to interface with specific classes of users or clients such as (1) patients, (2) HCPs, and (3) manufacturers. Portals can be designed to display and collect information from a designated subset of users. A software solution can operate on a common set of data but only allow access to an appropriate subset of the data that coincides with each class of user. In one embodiment, a cloud-based data hub can be used to collect the common data. Cloud data storage is a model of computer data storage in which the data can be centrally stored on servers and remotely accessed. With proper permissions, multiple devices or users can gain access to the remotely stored data including smartphones, tablets, laptops, desktops, or other computing devices. The central location of the data provides for remote access for multiple users. Each user or class of user can have different abilities to use the data (read, write, download, etc.).

Portals and/or interfaces can refer to data structures and protocols for exchanging data with a remote user or computing device. In one embodiment, a portal includes a web application provided by a server computing device over a network such as the internet. A client computing device (e.g., a patient device, a physician device, an HCP device) may access to the portal provided by the computing device by using a network address, such as a URL (uniform resource locator). The server computing device can be configured to host the web application portal, including transmitting web pages to the client computing device. In some embodiments, a portal includes a set of web pages and/or web page templates generated by a server computing device. In some embodiments, a portal may include alternate methods of exchanging structured data with a client computing device. For example, a portal may include a server computing device in network communication with a client application at a remote computing device. A remote computing device can generally include a computing device in network communication with a computing system, such as a server computing device.

Interfaces can include predefined data structures and layouts. A server computing device may use interfaces to standardize data communication with client computing devices and/or end-users. In one embodiment, an interface includes a webpage with a graphical user interface. The server computing device may populate the interface with data from a database to generate a webpage. The interface may define how the data is to be displayed and define how users may interact with the data and computer system. For example, the interface may include HTML and ECMAScript (e.g., JavaScript) code for displaying a webpage at a client computing device. In another embodiment, the interface may include a software application implementing operating system libraries to provide an interactive user interface at a client computing device. A portal and/or interface may be configured to receive user input (e.g., user actions), including prompting/requesting user input, and receiving user input or user actions. For example, a webpage may include a text input field, and a file upload component.

In alternate embodiments, an interface includes a programmatic application data interface, commonly referred to as an API or application interface. The application interface defines data structures and protocols for network communication between a client and server computing device. An application interface may include a web API based on HTTP (hypertext transfer protocol). More specifically, a server computing device may receive HTTP requests over a network, and respond by retrieving/modifying data stored in a database and/or executing software routines at the server computing device (e.g., processing/transforming stored data, generating/capturing data). The application interface may include any suitable communication standard, such as a network file system protocol, database connectivity protocol, stateful request protocol, stateless request protocol, and so on.

One method of manufacturing orthopedic implants includes the use of software designed to interface with a patient via a patient portal. In one embodiment, a patient portal provides access to data that is germane to patients. A patient portal can create or display patient identifiers that are linked to the patient, surgery, and implants to be used in surgery. Using the patient identifier or an electronic link, the patient can gain access the patient portal. Additionally, the patient portal can be configured to collect, confirm, or display other patient information (patient name, date of birth, gender, contact information, etc.). The patient portal can be constructed to receive image data directly from the patient via data input or upload. Another way of receiving information is to receive data from an external system, for instance, by communicating with hospital or office imaging systems, including PACS (Picture Archiving and Communication System). Furthermore, the patient portal can be constructed to receive consent from the patient relating to the use of image data in the design of the orthopedic implant. Additional useful features can include displaying of progress relating to delivery of the orthopedic implants.

One method of designing patient-specific interbody implants includes capturing important anatomical geometry and relative positioning using computed tomography (CT) or another imaging modality (MRI, simultaneous bi-planar radiography, etc.). The image data can be reconstructed into volumetric data containing voxels that are representative of anatomy.

One method of manufacturing orthopedic implants includes the use of software designed to interface with a physician or the office of the HCP, a HCP portal. In one embodiment, the physician has a unique identifier that can be linked to multiple patients under their care. Additionally, a physician may have an independent portal germane to their usage. Physicians and HCPs can have visibility to multiple patient data, workflows, and status to help provide improved patient care.

In one embodiment, the HCP portal allows a physician, nurse, physician assistant, nurse practitioner, or other party acting under the authority and direction of a HCP (such as office administrative staff) to gain access to relevant information. In one instance, a HCP portal can provide visibility to the designs of implants including, but not limited to, external envelope dimensional specifications (length, width, depth, coronal angle, sagittal angle, etc.), internal lattice specification, and implant stiffness. In one instance, an HCP portal can provide visibility to pathological anatomy, proposed correction plan and anatomy, anatomical metrics, and post-surgical image studies.

Furthermore, the HCP portal can provide an environment for granting approval of the treatment plan, including implant design. The portal can be used to collect, confirm or display information or solicit approval of treatments. The HCP portal can also be used by the physician to adjust or modify the prescription, treatment plan, or implant design based on their unique clinical appreciation of the pathological condition that requires surgery, or adjust or modify the proposed surgical plan. In one embodiment, physician approval of the treatment protocol or prescription, including the implant design, is required before manufacture and shipping from manufacturer. The HCP portal can provide an update of the workflow to help the HCP understand how the patient-specific implant is progressing through the design and manufacturing process.

One method of designing orthopedic implants includes the use of software designed to interface with the company, a company portal. The company portal can be configured to interface with design tools such as CAD software or imaging software. Design tools can be described as computer software used to design implants. Data can be extracted through the company portal and used with company software design tools to generate designs for implants. The implant designs can be returned to the data hub for display through the patient and HCP portals.

In one embodiment, the implant designs, treatment protocols, and resulting anatomical metrics can be displayed on the HCP portal for approval by the physician, surgeon, or authorized agent. After approval of the designs, protocols, and metrics, the company can manufacture the implant(s) to the specifications of the approved designs.

In some embodiments, a system generates the treatment protocol and manufactures the approved patient-specific implant. For example, a company can receive patient-specific request from HCP (accompanied with patient contact information and HCP contact information). The company can send an invitation to HCP that includes identifiers for HCP, patient, and prescription. The company may send an invitation to the patient that includes patient identifier at block 14. The patient may send to company their data including name, date of birth, gender, and image data. HCP sends case data including primary surgeon, surgery date, surgery location, pathology, and specific region(s) to be treated. The company can design implants based on patient scan data and/or surgeon case data. The company can send treatment protocol including images of patient anatomy, implant designs, and prescription to surgeon for approval. HCP may approve treatment protocol (plan) including implant designs. Approved implants are manufactured and sent to surgery.

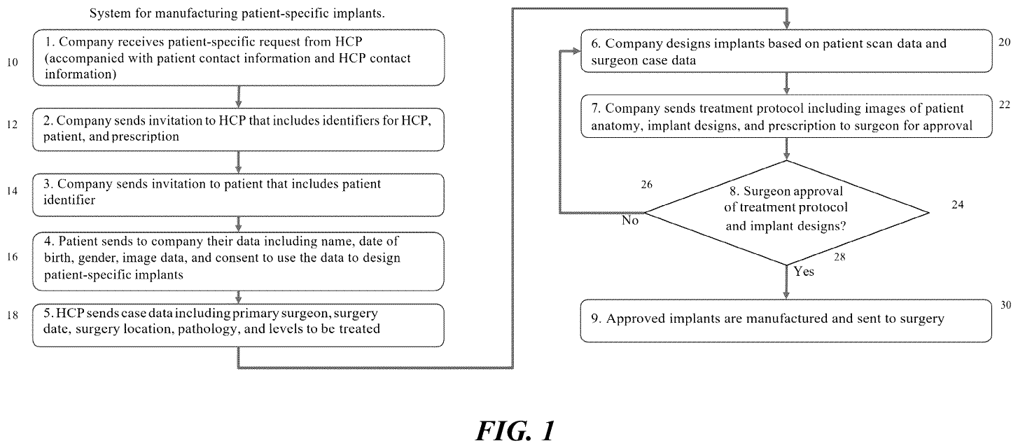

FIG. 1 shows a flow chart for designing and manufacturing patient-specific implants in accordance with one or more embodiments. In general, a system can include a collection of data from various blocks 10, 12, 14, 16, 18, designing the orthopedic implants using the data collected at block 20, presenting a provisional treatment protocol and designs for physician approval at block 24, and manufacturing the implants at block 30. If the provisional treatment protocol and implant designs are not approved by the physician, the company can adjust the protocol and implant designs based on physician feedback at 26. If the provisional treatment protocol is approved at 28, the implant design specifications can be used for manufacturing. One method of manufacturing can include additive manufacturing using Titanium alloy, metals, rigid plastics, or the like. Details of the system are discussed below.

At block 10, the system for manufacturing can receive a patient-specific request from a HCP. The request can include, without limitation, patient contact information, healthcare contact provider information, user account information, authorization information, or combinations thereof.

At block 12, the system can send one or more identifiers for the HCP, patient, prescriptions, medical device, or the like to the HCP. In some embodiments, the identifiers can indicate account(s) for healthcare provider, a user account for the patient, or the like.

At block 14, the system can send an invitation to the patient. The invitation can include the patient identifier, login information, account information, or the like and, in some embodiments, can include instructions for setting up a user account and linking the user account to healthcare provider. The patient identifier can include, without limitation, one or more electronic links for setting up a user account, providing access to an account, etc.

At block 16, the patient can provide the system with data, including the patient's name, date of birth, gender, image data, and/or consent to use the data, or other data, such as data provided by the healthcare provider directly to the system. The patient can review data prior to approval and approve use of all the data or a subset of data. The patient data can be provided via email, an upload portal, FTP site, or the like. The HCP can send information or notifications to the patient to facilitate the patient's response to the invitation. In some embodiments, the HCP can send the invitation from the system directly to the patient via an SMS (Short Message Service), emails, etc. The patient can access the patient user account to manage data (including data sent from the patient, data sent from healthcare providers, or the like), settings (e.g., security settings), permissions, or the like. The user account can manage permissions for multiple healthcare providers, physicians, or individuals (e.g., family members) associated with the patient.

At block 18, the HCP can send data, including, without limitation, physician information (e.g., primary surgeon), procedure information (e.g., surgery date, surgery location, hospital information, etc.), pathology, treatment information (e.g., levels to be treated for spine surgeries), or the like. The data can be associated with the identified patient. In some embodiments, the data can include information from clinicians. The clinician information can include surgeon preferences, including preferred delivery instruments, preferred parameters for implants, or the like.

At block 20, the system can design one or more implants based on the patient data and/or surgeon case data. The surgeon case data can include symptom information, a diagnosis, and treatment information. The surgeon case data can be linked with the patient data.

At block 22, a treatment protocol can be sent to the healthcare provider for review, comment, and approval. The treatment protocol can include, without limitation, one or more images of the patient anatomy, implant designs, and prescription. The images of the patient anatomy can be annotated by the company to indicate implantation sites, surgical approaches, and anatomical features of interest, such as significant anatomical anomalies. This can assist with physician review. In some procedures, the treatment protocol can be sent to a hospital, which in turn distributes the treatment protocol to one or more members of a surgical team. The surgical team can review, revise, and approve, via healthcare portal, or the treatment protocol. In some embodiments, the system can generate post-treatment images illustrating the predicted post-treatment position of anatomical features. The physician can use the post-treatment images to assess the expected outcome and predict efficacy. The post-treatment images can be included in the treatment protocol or can be included in a post-treatment report.

At block 24, if the healthcare provider approves the treatment protocol and implant designs, the system can automatically begin the manufacturing process. In some embodiments, a surgeon can improve the treatment protocol and implant designs while also providing additional feedback that is incorporated into the implant design. For example, the surgeon can provide additional dimension/configuration, treatment input, or other information suitable for designing the implant. In some instances, the healthcare provider may acquire additional images the patient used in the approval process. The additional data can be sent to the company to update the design and/or treatment protocol.

If the surgeon rejects the proposed design and/or treatment protocol at 26, the surgeon can provide input for redesigning the implant. The system can update the implant design based on the input and can send the updated design and treatment protocols to the surgeon at block 22. This process can be repeated until the design and/or treatment protocol are approved.

Other embodiments may include instances where users (patients, physicians, HCP offices, etc.) do not need to provide the information using the software interface, but rather confirm the accuracy of collected data.

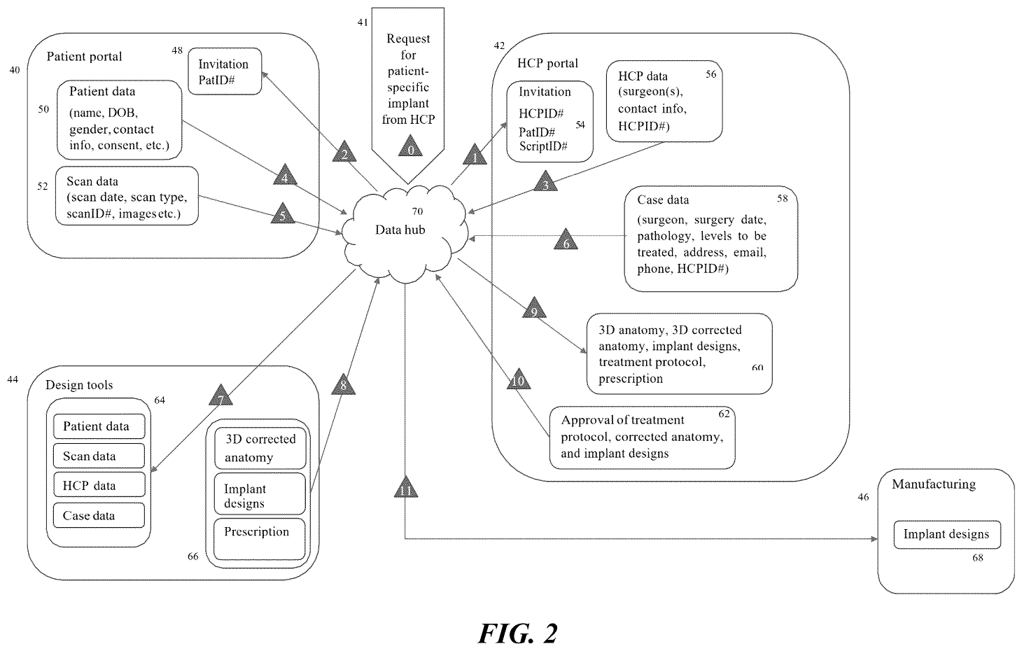

FIG. 2 shows a diagram describing information flow within a system for designing and manufacturing a patient-specific implant. The diagram is divided into portals 40, 42, 44 that are configured to facilitate information collection, exchange, and display based on the class of user. Additionally, a central database or data hub 70 (“data hub 70”) can be configured to collect data and provide access to each user based on their class and permissions. A manufacturing section 46 represent a facility or machine configured to manufacture implants based on design specifications.

Each of the users are issued unique identifiers to allow for tracking of workflow, identification purposes, and traceability required for implantable devices. Other user information including names, date of birth, gender, and contact information may be helpful for data management purposes.

The process is initiated by a request for a patient-specific implant originating from the physician or HCP 41. After request 41 is received by the data hub, an invitation is sent to the HCP that includes issues or confirms a HCP identifier.

Patient portal 40 is configured to interface with the patient and facilitate collection or confirmation of patient-related data. In one embodiment, data hub 70 is configured to receive a request for a patient-specific orthopedic implant. Data hub 70 can be configured to send and receive data. Data hub 70 sends a message 54 to HCP via HCP portal 42 which confirms or assigns a HCP identifier (HCPID #), patient identifier (PatID #), and prescription identifier (ScriptID #). Data hub 70 sends a message to patient via patient portal 40 which confirms or assigns a patient identifier. If not already known, HCP sends data 56 to data hub 70 that may include affiliated surgeons. If not already known, patient sends patient data 50 to data hub 70. Patient sends scan data including images used for diagnostic purposes, date of image scan, type of image scan (typically in the DICOM image format), and patient consent to use such data in the design and manufacture of implants. Additionally, the patient may consent to use of their data for compiling within a database to aid with future treatments. Using HCP portal 42, HCP provides additional data 58 about the case including primary surgeon, surgery date, pathology, diagnosis, specific regions to be treated, and other information. After collection of all data 48, 50, 52, 54, 56, 58 within data hub 70, company can begin design of patient-specific implants. Using design tools 44, data collected via patient portal 40, and data collected via HCP portal 42, design of patient-specific implants can begin. Upon design of implants, including adjustment of anatomy from a pathological state to a corrected state, the provisional treatment protocol can be submitted through data hub 70 to the physician or HCP for approval within the HCP portal. Upon approval of treatment protocol 62, implant design specifications 68 are sent to manufacturing 46.

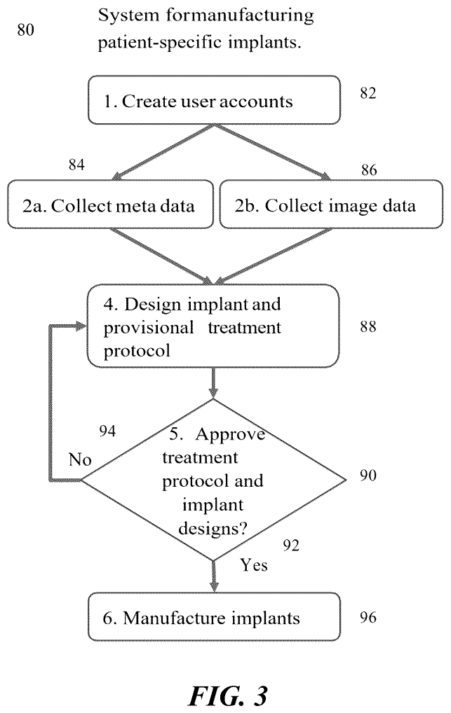

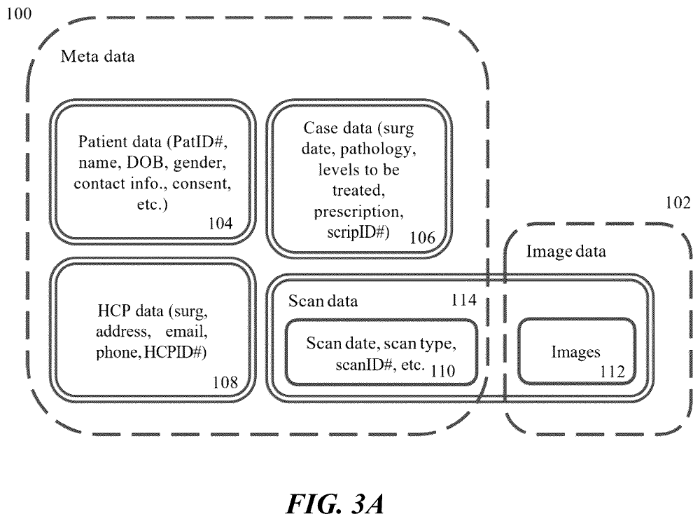

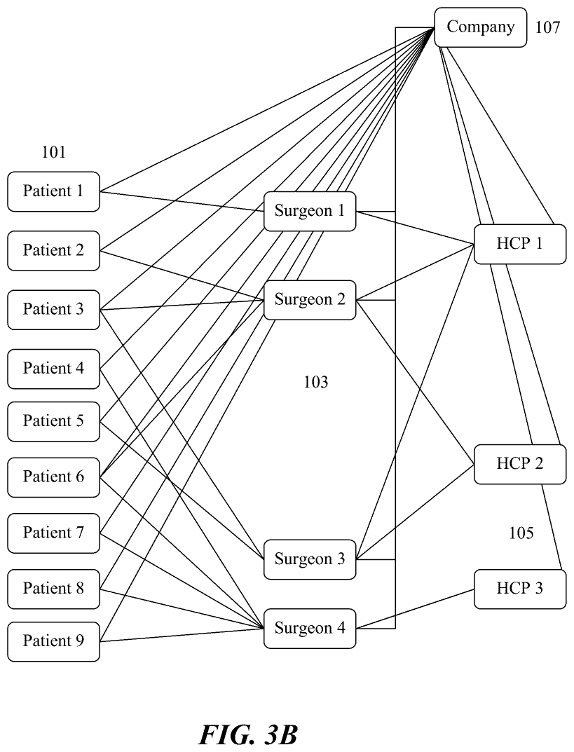

FIG. 3 shows a flow diagram 80 describing one embodiment of a system of designing and manufacturing patient-specific implants. In one embodiment, user accounts can be created 82 to provide a structure for data to be collected and linked. Typical users include patients and HCPs. System 80 can have multiple users based on the use of information within system 80. The HCP user group can be divided into different classes of users. For example, a surgeon may have different access and permissions than physician office personnel.

Following the creation of user accounts 82, collection of meta data 84 and collection image data 86 can begin. Meta data includes data that is used to describe other data, in this case, meta data represents data identifying patient, surgeon, case, and scan. Image data can be described as data representative of patient anatomy. In one scenario, image data can be collected as adjacent two-dimensional slices of cross-sectional anatomy. Consecutive two-dimensional cross-sectional images can be compiled into thee-dimensions to form a three-dimensional representation of anatomy.

After meta and image data are collected 84, 86 the data can be combined and used to design a provisional treatment protocol and associated implants 88. Following creation of a provisional treatment protocol and implant designs, a physician can be commissioned to provide approval of the protocol and implant designs. If protocol and designs are not in condition for approval 94, the surgeon can work with the company to devise an appropriate protocol and designs 88. Following approval, the implant designs are sent to manufacturing 96 and ultimately delivered to surgery for implantation into the patient.

FIG. 3A shows a diagram categorizing data to be used in the design and manufacture of patient-specific implants. This data is typically comprised of meta data 100 and image data 102. Meta data 100 can contain information such as patient identification 104 (name, contact information, date of birth, gender, acceptance of consent, etc.), HCP identification 108 (surgeon name, contact information, etc.), case data 106 (surgery date, pathology, diagnosis, treatment levels, etc.) and scan data 114 (scan date, scan type, DICOM data, images, etc.). Image data 102 can contain data related to the diagnostic images 112 of the patient obtained and collected by the HCP. Images 112 can be formatted as a three-dimensional collection of two-dimensional slices (typical to a CT or MRI volumetric scan). Scan data 110 can contain data identifying the scan date, scan type, scan settings, and DICOM data.

FIG. 3B shows a diagram showing relationships between users. The lines between users represent data that can be shared between users. In one embodiment, patients 101 are linked to one or more surgeons 103 and company 107. Surgeons 103 are linked to one or more patients 101, one or more HCP offices 105, and company 107. HCP offices 105 can be linked to one or more surgeons 103 and company 107. Company 107 is linked to all users; the company requires access to all of the information in order to design the surgical plan and deliver implants to surgery.

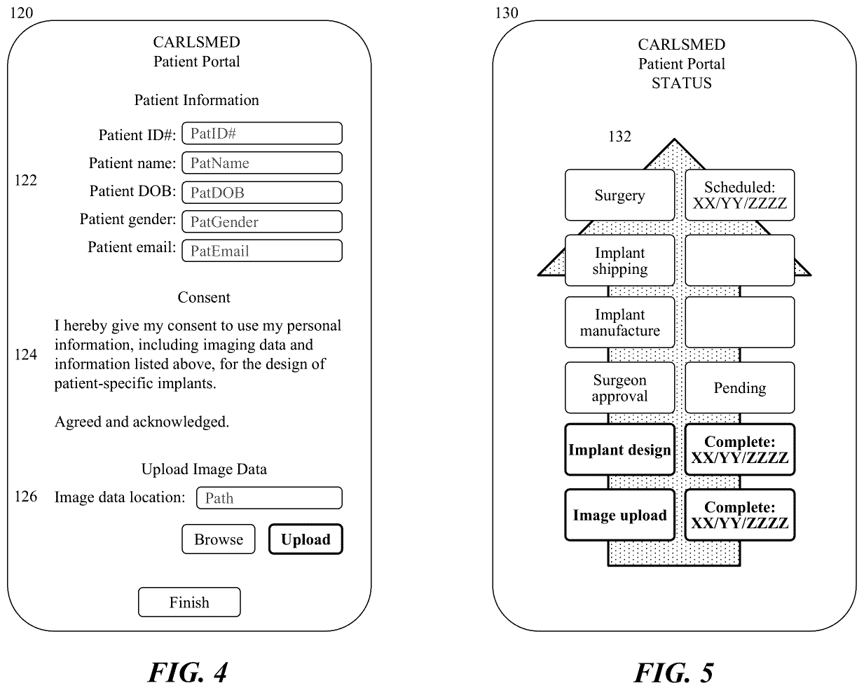

FIG. 4 shows one view of an embodiment of a patient portal interface 120. In this embodiment, the patient could be prompted to enter or confirm relevant information to aid with unique identification (patient identifier, name, date of birth, gender, email, etc.) 122. One further aspect of this interface is the consent section 124 asking for permission from the patient to use their data for design of the orthopedic implant. In another embodiment, patient consent can be granted to allow collection of anonymized demographic data and outcomes to help refine future treatment protocols. Another aspect of the interface provides for upload of digital imaging data 126.

FIG. 5 shows another view of a patient portal interface 130. In this view, a status graphic 132 is displayed to provide information to the patient regarding the status of the design, manufacture, and delivery of the orthopedic implant.

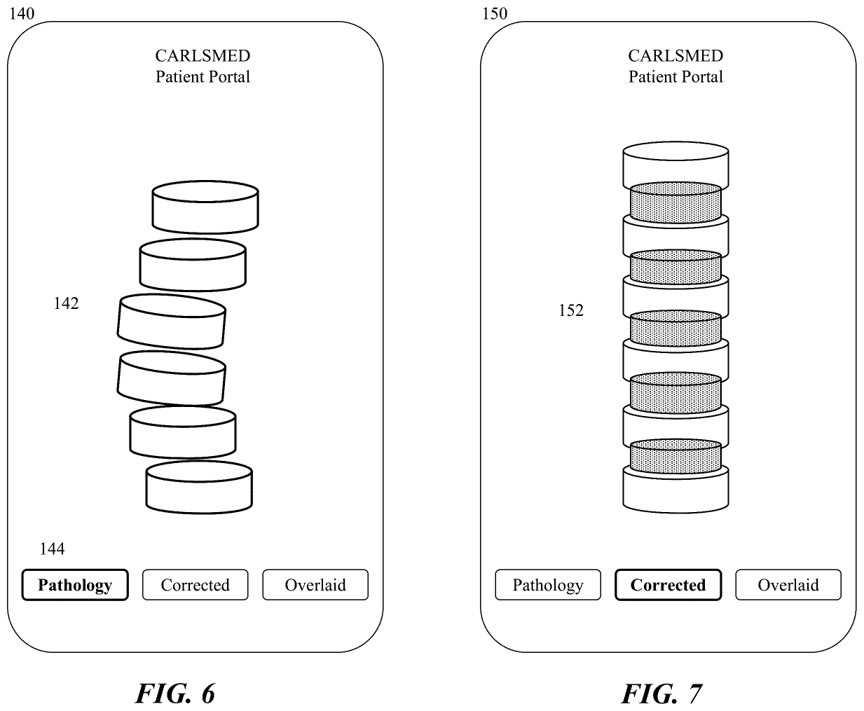

FIG. 6 shows another view of a patient portal interface 140. In this view, image(s) or a virtual model representing patient anatomy containing the pathological condition is displayed 142. The present image shows representations of a patient's spinal anatomy. The image(s) or models can be displayed and manipulated using typical touch-screen gestures including zoom, pan, and rotate. Virtual ‘buttons’ 144 below the image can be used to toggle between pathology captured by the pre-operative patient scan (pathological anatomy), corrected anatomy based on a treatment protocol, and a composite model showing both the pathological anatomy and the treatment protocol overlaid upon each other.

FIG. 7 shows another view of a patient portal interface 150. In this view, an image(s) or a virtual model representing corrected anatomy 152 as described by a treatment protocol is displayed.

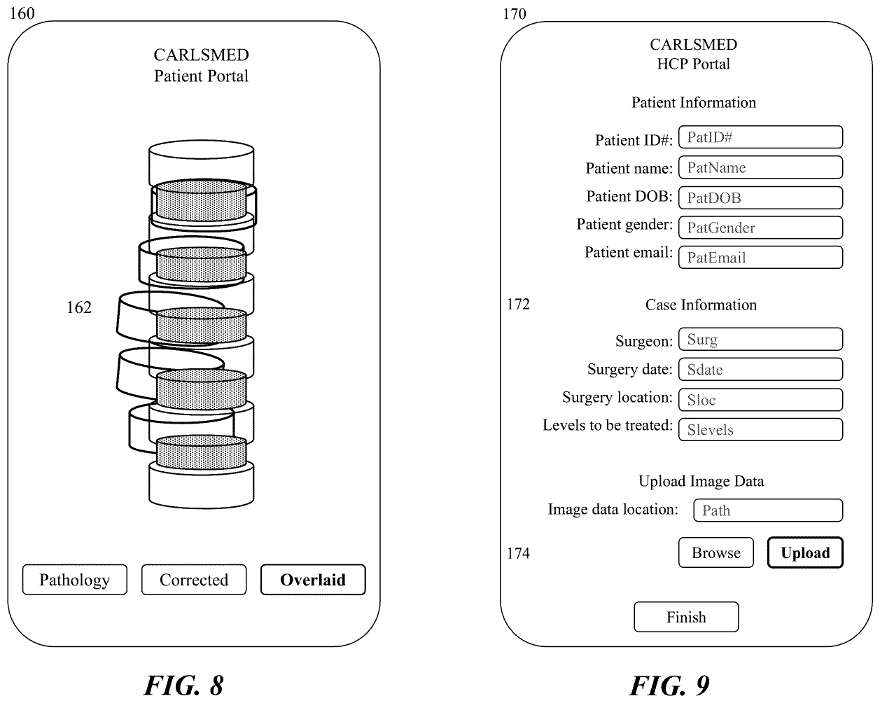

FIG. 8 shows another view of a patient portal interface 160. In this embodiment, an image(s) or a virtual model representing a composite of both the pathological anatomy and the treatment protocol 162.

Each of the portals can be configured to provide different views and functionality for the different users or class of users. In the present embodiment, the views and functionalities within the patient portal shown in FIGS. 4 through 8 are different than the views of the HCP portal shown in FIGS. 9 through 16.

FIG. 9 shows one view of one embodiment of a HCP portal interface 170. In this embodiment, there is a section or sections 172 for a HCP to enter or confirm relevant information to aid with unique identification (patient identifier, patient name, patient date of birth, patient gender, patient email, primary surgeon, surgery date, surgery location, levels to be treated, etc.). Another section of the interface provides for upload of digital imaging data 174.

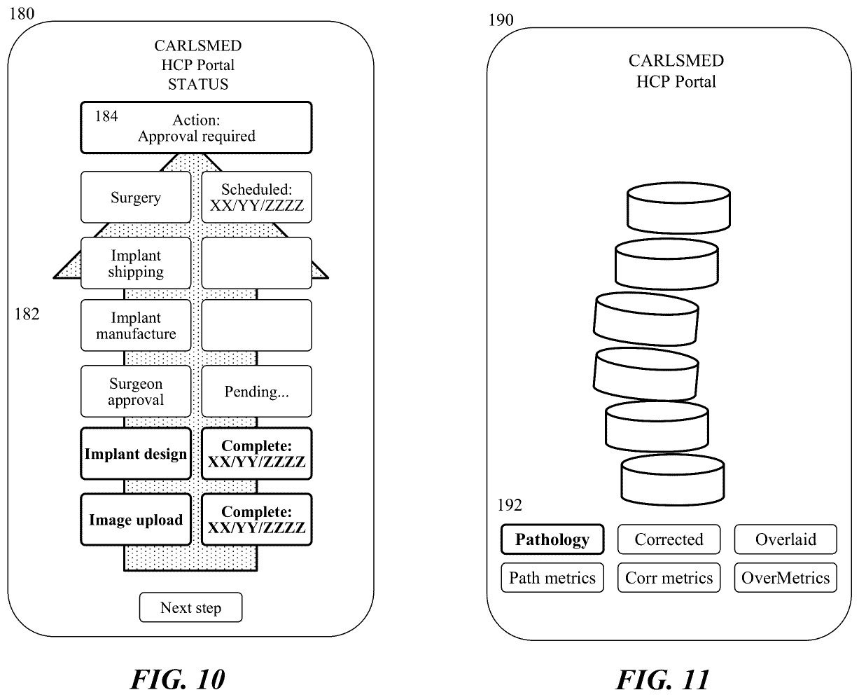

FIG. 10 shows another view of a HCP portal interface 180. In this view, a status interface 182 is displayed to provide information to the patient regarding the status of the design, manufacture, and delivery of the orthopedic implant. Presently, there is a notification regarding the status 184; in one instance, a HCP may be prompted by notification to review and approve treatment protocol as suggested. If approval is granted, the implant designs can move to manufacturing. If the protocol is not approved, suggested modifications from the surgeon can be incorporated, implants redesigned, and another notification sent to the HCP alerting them of pending approval of a different treatment protocol and implant designs.

FIGS. 11 through 13 shows additional views of a HCP portal 190, 200, 210. In these views (as in FIGS. 6 through 8), image(s) or a virtual model representing patient anatomy is displayed. FIG. 11 shows representations of a patient's spinal anatomy containing the pathological condition, in this embodiment, a deformed spine. The image(s) or models can be displayed and manipulated using typical touch-screen gestures including zoom, pan, and rotate.

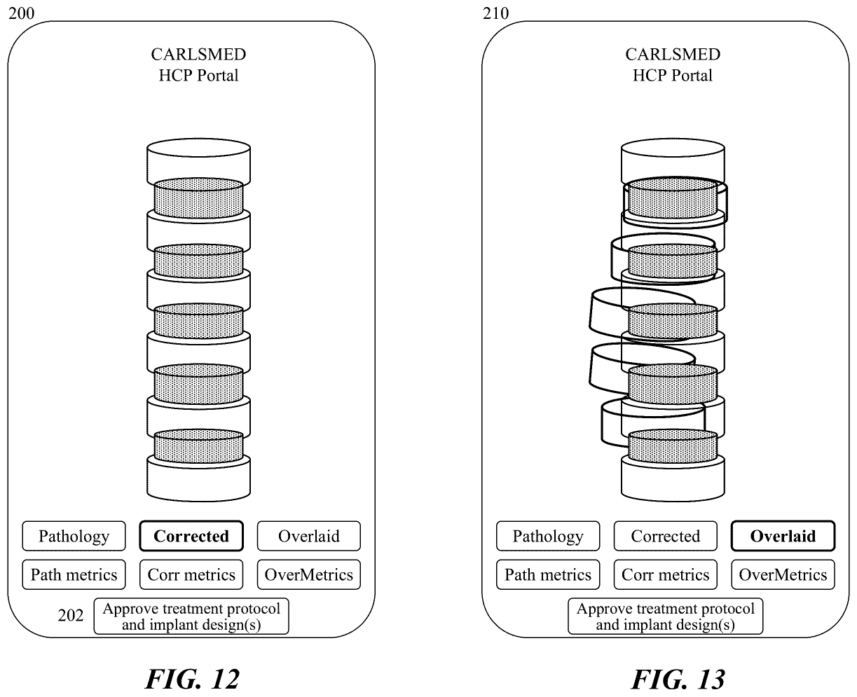

In these views of the HCP portal, section 192 contains several virtual ‘buttons’ that can be seen beneath the displayed anatomical model. Additional or different ‘buttons’ and the underlying functionality are present in different views and portals for different users; in the present embodiment, the ‘buttons’ for the HCP portal are different than those displayed on the patient portal (FIGS. 6 through 8). FIG. 12 displays a ‘button’ 202 that provides a physician the opportunity to approve the treatment protocol and implant designs.

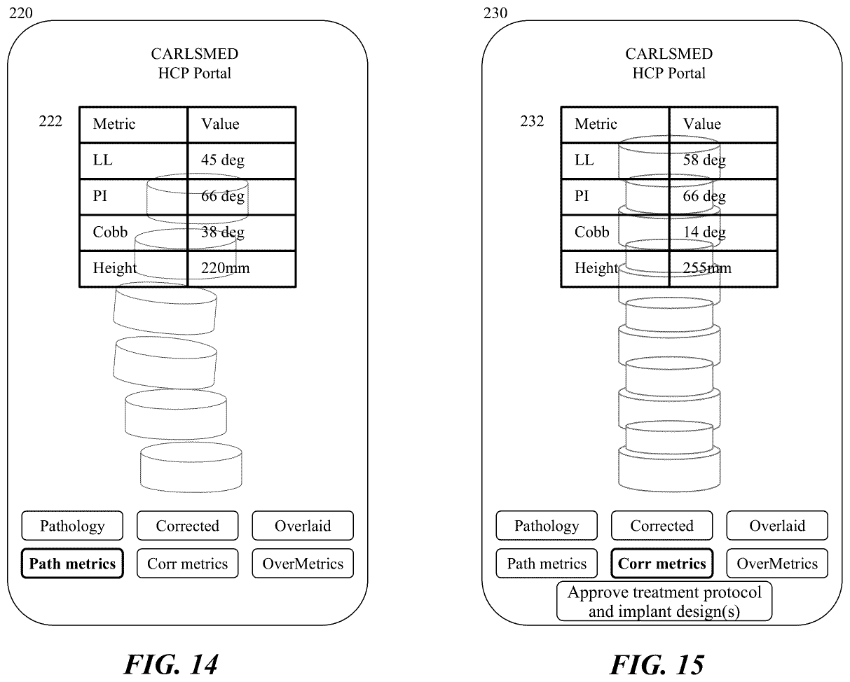

FIG. 14 shows another view of an embodiment of a HCP portal interface. In view 220, table 222 displays relevant anatomical metrics typically used in the analysis and treatment of a spinal deformity. However, other metrics may be used to assess different orthopedic pathologies. In the present embodiment, lumbar lordosis (LL), pelvic incidence (PI), Cobb angle (Cobb), and height are displayed for the subject pathological anatomy.

FIG. 15 shows another view of an embodiment of a HCP portal interface. In view 230, table 232 displays the metrics representative of the corrected anatomy. The corrected anatomy is a result of the provisional treatment protocol.

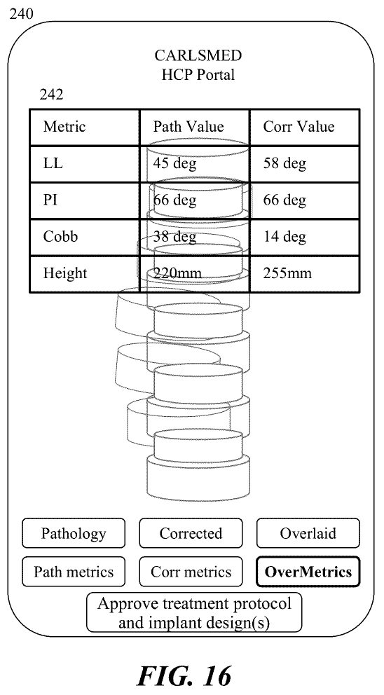

FIG. 16 shows another view of an embodiment of a HCP portal interface. In view 240, table 242 allows for direct comparison of pathological and corrected metrics as a result of the provisional treatment protocol. These metrics are used by physicians to help assess the pathology and the provisional treatment protocol. The physician uses these metrics to help determine if the provisional treatment protocol can be approved to correct the pathology.

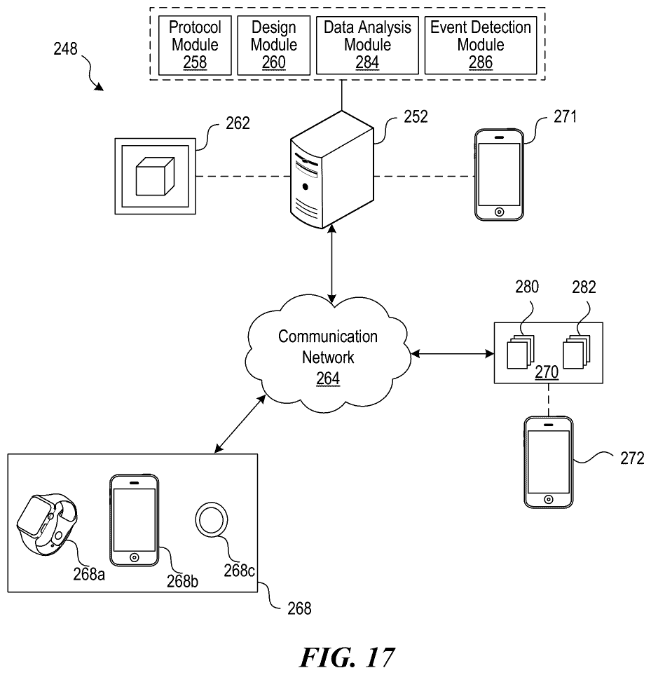

FIG. 17 illustrates a system 248 for monitoring patients according to an embodiment. In some embodiments, a computing system 252 can create a user account associated with a patient. The system 252 may include a single server computing device, or multiple computing devices. The system 252 can also receive data from one or more patient devices 268, a user device 272 (e.g., a computing device), and/or a healthcare provider (HCP) 270. The patient devices 268 can include a smartphone device 268b, a wearable connected device, such as an activity monitor, a smartwatch 268a, a smart ring 268c, or any connected device that can collect, for example, biometric data, activity data, body posture data, spine curvature data, heart rate data, oxygen saturation level data, breathing data, stress levels, data transmitted by the implant (e.g., loading data, motion data from an artificial disc, etc.), or the like. The patient devices 268 can be, for example, a wearable smartwatch 268a that can collect exercise data, body temperature data, heart rate data, position data (e.g., Global Positioning System (GPS) data), and other user information. The patient devices 268 can collect data before, during, and/or after one or more monitoring periods, such as a recovery period, scheduled period, therapy period, etc. The patient devices 268 can be a connected scale (e.g., a smart-scale) configured to track patient body mass, weight, etc. In some embodiments, the patient devices 268 can collect data before, during, and/or after the implant design process. The patient devices 268 can communicate via, for example, a local area network, a broad area network, direct connection, or the like.

The system 248 can design a patient-specific orthopedic implant based on the received data, and a manufacturing system 262 can produce the implant according to the design. The system 248 can also generate treatment protocols, which can include an entire surgical technique or portions thereof. The implant can be configured for the patient's anatomy as discussed in connection with FIGS. 1-16.

In robotic-assisted procedures, the robotic instructions from the system 248 can be used to control to a robotic apparatus (e.g., robotic surgery systems, navigation systems, etc.) for an implant surgery or by generating suggestions for medical device configurations to be used in surgery. In some procedures, both manual and robotic procedures can be performed. For example, one step of a surgical procedure can be manually performed by a surgeon and another step of the procedure can be performed by a robotic apparatus. Additionally, patient-specific components can be used with standard components made by the system 248. For example, in a spinal surgery, a pedicle screw kit can include both standard components and patient-specific customized components. For example, the implants (e.g., screws, screw holders, rods, etc.) can be designed and manufactured for the patient and the instruments can be standard instruments. This allows the components that are implanted to be designed and manufactured based on the patient's anatomy, and/or surgeon's preferences to enhance treatment. The patient-specific devices can improve, without limitation, delivery into the patient's body, placement at the treatment site, and interaction with the patient's anatomy.

The progress of treatment can be monitored over a period of time to help update the system 248. In trainable systems 248, post-treatment data can be used to train machine learning (ML) programs for developing surgical plans, patient-specific technology, or combinations thereof. Surgical plans can provide one or more characteristics of at least one medical device, surgical techniques, imaging techniques, etc.

With continued reference to FIG. 17, the system 248 may be connected to one or more communication networks 264. The communication network 264 may further be connected with the system 252, the patient device 268, and the HCP 270. The patient device 268 can send data to the system 252 via the network 264 and can provide access to patient portals. For example, the patient device 268 can display the patient portals discussed in connection with FIGS. 4-8. The HCP 270 can send patient data sets 280, 282 and provide access to HCP portals via a physician device 272. The communication network 264 may be a wired and/or a wireless network. The communication network 264, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and other communication techniques known in the art.

The system 252 may be implemented as a facility over “the cloud” and may include a group of modules. More specifically, system 252 may include a server computing device, or multiple computing devices in a distributed computing system. The group of modules may include a data analysis module 284, protocol module 258, and a design module 260. The system 252 can use one or more segmentation algorithms to process images. The segmentation algorithms can include, without limitation, one or more edge detection algorithms, boundary detection algorithms, thresholding, and other image processing algorithms and techniques applied to images. Anatomical elements or features in images (e.g., scans, digital images, etc.) can be identified after segmentation algorithms are used to segment features in images of the patient. The patient data can be imported into a modeling program to generate a CAD model of the patient's anatomy, including pre- and post-treatment models. The patient-specific CAD model can be used to generate an implant CAD model. A treatment CAD model can include both the patient-specific CAD model and the implant CAD model to allow for manipulation of the implant design, position of anatomical elements, or the like. The system 252 can convert the implant CAD model to manufacturing data.

The system 252 can be configured to receive one or more patient data sets that can include, without limitation, data representative of the patient's condition, anatomy, pathology, medical history, preferences, recovery plan, and/or any other information or parameters relevant to the patient. For example, the patient data set can include medical history, surgical intervention data, treatment outcome data, progress data (e.g., physician notes), patient feedback (e.g., feedback acquired using quality of life questionnaires, surveys), biometric data, exercise data, clinical data, provider information (e.g., physician, hospital, surgical team), patient information (e.g., demographics, sex, age, height, weight, type of pathology, occupation, activity level, tissue information, health rating, comorbidities, health-related quality of life (HRQL)), vital signs, diagnostic results, medication information, allergies, image data (e.g., camera images, Magnetic Resonance Imaging (MRI) images, ultrasound images, Computerized Axial Tomography (CAT) scan images, Positron Emission Tomography (PET) images, X-ray images), diagnostic equipment information (e.g., manufacturer, model number, specifications, user-selected settings/configurations, etc.), or the like. In some embodiments, the patient data set includes data representing one or more of pain information, workout history, daily or weekly exercise information, patient identification number (ID), age, gender, body mass index (BMI), spinal metrics (e.g., lumbar lordosis, Cobb angle(s), pelvic incidence, etc.), disc height, segment flexibility, bone quality, rotational displacement, and/or treatment level of the spine. In some embodiments, the patient data sets 280, 282 include externally generated data, third-party data collected from external sources (e.g., remote servers, databases, websites, journals, articles, platforms, etc.), databases, software program platforms, navigation systems, treatment planning platforms, or combinations thereof. The databases can store, for example, machine learning modules, algorithms, models (e.g., anatomical models), etc.

The client computing device can communicate with one or more remote software program platforms and can store plans, medical records, virtual models, etc. The virtual models can include metrics and can include, for example, two-dimensional (2D) models, three-dimensional (3D) models, or other models of one or more anatomical elements, implants, navigation equipment, and/or instruments. A virtual model can include anatomical elements (e.g., vertebral bodies, ligaments, spinal segments, etc.), simulations, biometrics, etc. For example, the system 252 can authenticate and receive data from authenticated software programs or platforms and use the received data to generate plans, including treatment plans, recovery plans, tracking plans, etc. In some embodiments, the system 252 can be periodically or continuously synchronized with the third-party data source. The third-party data source can include a CAD platform or application that updates models, and the updated models can be automatically sent to, or retrieved by, the system 252. A client computing device or server can validate the third-party model for analyzing, planning, and/or generating predictions. For example, the third-party model can be validated by authenticating the third-party data source, translating the third-party model into a readable digital format, modifying the third-party virtual model for importation into an existing model, confirming that the imported third-party virtual model is compatible with other model elements, and/or importing all or a portion of the third-party virtual model and associated data (e.g., spinopelvic metrics or parameters). The imported third-party virtual model can be, for example, a 2D model, a 3D model, or other model of one or more anatomical elements, implants, navigation equipment, and/or instruments.

The system 252 can include modules (e.g., patient monitoring platforms, trained event detection modules, etc.) that communicate with the third-party data source such that the third-party data source modifies, replaces, or adjusts the data. In some embodiments, the client computing device can be periodically or continuously synchronized with third-party treatment planning software, CAD software, navigation software, devices 268, 271, 272 (e.g., smartphones or tablets capturing images of the surgery site), a robotic surgery system, or the like. In some embodiments, the synchronization can be performed based on one or more synchronization triggers, including modification of data or event (e.g., publication of article(s), planned manufacturing of implant, start of surgery, etc.). In some embodiments, a user can set a schedule (e.g., daily, weekly, monthly, etc.) for synchronization using a mobile application.

The system 252 can combine anatomical models from multiple third-party data sources to generate a multi-source anatomical model. The system 252 can analyze, score, rank, and/or modify the multi-source anatomical model to, for example, match patient images, generate predictions, generate surgical plans, etc. The anatomical models can include standard elements/models modifiable (e.g., scaled, sized, or altered to match patient data) based on patient data, patient-specific models with topology matching patient topology, etc. For example, the system 252 can combine one or more standard elements/models of anatomy, patient-specific models, models of implants, and other models to generate multi-source models. In some embodiments, the standard elements/models can be, for example, standard or generic vertebrae positioned to represent the patient's spine. The system 252 can retrieve the standard elements/models from a library or database. In some embodiments, the system 252 selects a standard element/model based on the patient's age, gender, body type, etc. The elements/models can be positioned relative to one another based on patient measurements to model positional relationships between anatomical elements, thereby creating a patient-specific model utilizing standard or generic anatomical elements. A virtual location model of anatomy can be displayed for viewing by a user. In some embodiments, a model includes both standard elements/models and patient-specific elements/models. The standard elements/models can be used when an insufficient amount of information is available to generate patient-specific elements/models.

The multi-source models can be location models, topology models, and location-topology models. The location models can represent the anatomical positions of the patient anatomy and can include, for example, standard anatomical elements, patient-specific anatomical elements, and/or features disclosed herein. The topology models can represent the topology of the patient's anatomy. When an insufficient amount of information is available to generate a topology (e.g., an entire vertebral endplate), the system 252 can generate approximated topologies using standard topologies, machine learning modules, etc. The location-topology models can represent both the position and the topology of the patient's anatomy. In some embodiments, the multi-source models can be 2D models, 3D models, etc. The system 252 can include import and export modules, and the third-party data source can also include one or more design platforms. The system 252 and/or user can select metrics and/or parameters for the multi-source models.

The system 252 can combine treatment plans from multiple third-party data sources to generate multi-source treatment plans. In some embodiments, the system 252 can receive multiple treatment plans and integrate the plans together to generate a single multi-source treatment plan. The system 252 can select a set of metrics from a first treatment plan and a second set of metrics from a second treatment plan. A multi-source treatment plan and a virtual anatomical model can be generated to achieve both the first and second sets of metrics. For example, the system 252 can generate a multi-treatment recovery plan based on the multiple individual treatment plans, such as an anterior fusion plan (e.g., a lumbar fusion plan for implanting one or more cages) and a posterior fusion plan (e.g., a fusion plan for implanting one or more rods). The multi-treatment recovery plan can be staged based on status of fusion (e.g., percentage of fusion, completed fusion, etc.), time after surgery, recovery metrics, etc. The multi-treatment recovery plan can simulate long-term outcomes associated with each treatment, spine pathology, and the like. The multi-treatment recovery plan can be synchronized to update information (e.g., metrics, types of visual images, etc.) selected by, for example, a physician, healthcare provider, system 252, machine learning module, or the like. The metrics and/or parameters can be from different models and/or plans. In some embodiments, the multi-treatment recovery plan can be updated based on modification(s) to a linked third-party source that provided a plan.

In some embodiments, the system 252 can be a third-party data source. The description of third-party data sources applies to the system 252 unless indicated otherwise. For example, a user can select whether the system 252 generates plans and/or acts as a third-party data source supporting a remote system that generates plans. In some third-party data source embodiments, the system 252 can collect data, generate treatment data (e.g., models, predictions, corrected pathologies, navigation data, metrics, etc.) based on the collected data, and send the treatment/recovery data to a remote system that generates, based on the recovery data, one or more recovery plans, implant devices, etc. A user can review, revise, and/or approve recovery data and/or plans during this process.

The system 252 can include and/or communicate with a data analysis module 284 that is configured with one or more algorithms for identifying a subset of the third-party data from the database that is likely to be useful in developing a patient-specific plan (e.g., treatment plan, recovery plan, or another plan). For example, the data analysis module 284 can compare patient-specific data (e.g., the patient data sets 280, 282 received from the HCP 270) to the third-party data from a database (e.g., the third-party data sets) to identify similar data (e.g., one or more similar patient data sets in the third-party data sets). The comparison can be based on one or more metrics/parameters, such as age, gender, BMI, lumbar lordosis, pelvic incidence, and/or treatment levels. The metric(s)/parameter(s) can be used to calculate a similarity score for each reference patient. The similarity score can represent a statistical correlation between the patient data set and the reference patient data set. Accordingly, similar patients can be identified based on whether the similarity score is above, below, or at a specified threshold value. For example, as described in greater detail below, the comparison can be performed by assigning values to each metric/parameter and determining the aggregate difference between the subject patient and each reference patient. Reference patients whose aggregate difference is below a threshold can be considered as similar patients.

The data analysis module 284 can further be configured with one or more algorithms to select a subset of the third-party patient data sets, e.g., based on similarity to the patient data set and/or treatment outcome of the corresponding reference patient. For example, the data analysis module 284 can identify one or more similar patient data sets in the third-party data sets, and then select a subset of the similar patient data sets (e.g., a subset including portions of multiple data sets or a subset of entire data sets) based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 284 calculates an outcome score by assigning values to each outcome parameter. A patient can be considered to have a favorable outcome if the outcome score is above, below, or at a specified threshold value. A user can set the threshold value, or the data analysis module 284 can calculate the threshold value. If the outcome score meets retraining criteria (e.g., outcome score not meeting an accuracy score), machine learning module(s) can be retrained using the patient data. The retraining criteria can be inputted by a user, calculated based on accuracy of the confidence scoring algorithms, or the like.

In some embodiments, the data analysis module 284 selects a subset of the third-party data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively. In some embodiments, the data analysis module 284 can identify similarities and/or differences between the third-party data and the patient data. For example, similar spine metrics can be highlighted and correlated in tables. A user can visually identify the number and types of correlated data to determine whether sufficient similarities exist between the third-party data and the patient data. In some embodiments, the user can input information for determining similarities and/or outcome parameters.

The protocol module 258 can apply one or more algorithms to correct anatomy, develop implant designs, select implant designs, provide prescriptions, or the like. The protocol module 258 can serve as a data hub that collects information and store images of patients and types of implants, required in spinal surgeries. In some implementations, a similar module can be used for other types of surgeries to, for example, store patient data, device information, etc. The images stored by the protocol module 258 may be any of scans, camera images, Magnetic Resonance Imaging (MRI) images, ultrasound images, Computerized Axial Tomography (CAT) scan images, Positron Emission Tomography (PET) images, and X-ray images. In one case, the images may be analyzed to identify anatomical features, abnormalities, and salient features in the images, for performing spinal surgeries on the patients. In some implementations, the protocol module 258 can store additional implant surgery information, such as patient information (e.g., sex, age, height, weight, type of pathology, occupation, activity level, tissue information, health rating, etc.), specifics of implant systems (e.g., types and dimensions), availability of available implants, aspects of a surgeon's preoperative plan (e.g., surgeon's initial implant configuration, detection and measurement of the patient's anatomy on images, etc.), etc. In some implementations, the protocol module 258 can convert the implant surgery information into formats useable for implant suggestion models and algorithms. For example, the implant surgery information can be tagged with particular identifiers for formulas or can be converted into numerical representations suitable for supplying to a machine learning model. The protocol module 258 may include design tools. The design tools can be used to measure distances between a number of salient features of one vertebra with salient features of another vertebra, for identifying implantation sites, spaces, disk pinches, bulges, etc. If the spinal abnormalities are identified, the protocol module 258 may graphically identify areas having the spinal abnormalities and may send such information to the HCP 270. The HCP 270 can respond to requests for additional information.

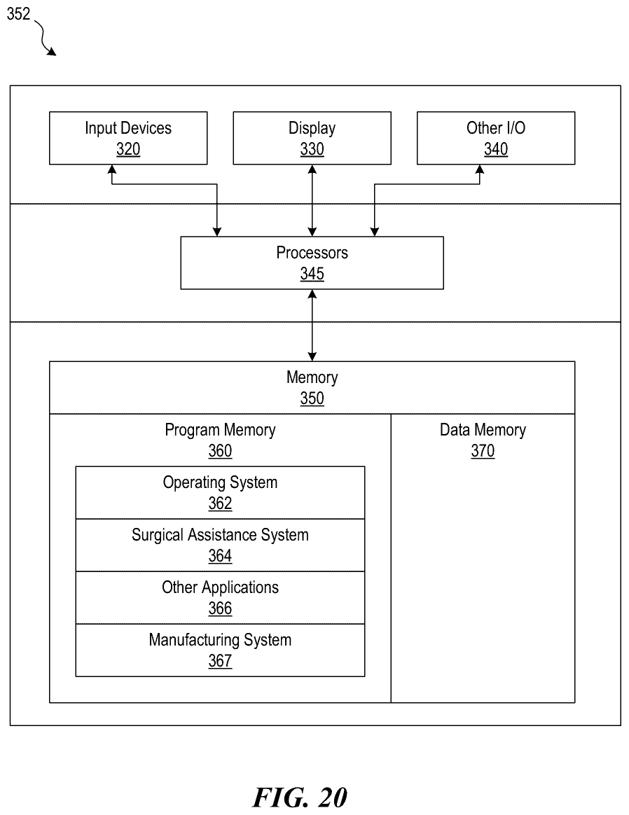

The design module 260 can generate representations of anatomy, CAD models, manufacturing programs or instructions, segmentation tools, segmentation algorithms, etc. Output from the design module can be provided to the protocol module 258. The protocol module 258 and the design module 260 can work together to generate designs for implants and treatment protocols optimized for patient-specific implant designs. In some embodiments, the design module 260 can generate one or more virtual models, tool paths, instruction sets, or the like for manufacturing. In some embodiments, the system 252 can form the methods discussed in connection with FIG. 1 and can include one or more systems discussed in connection with FIG. 20. For example, the protocol module 258 and the design module 260 can be components of the surgical assist system discussed in connection with FIG. 20.

The manufacturing system 262 can receive manufacturing data from the system 252. The manufacturing data can include virtual model data, tool path data, instruction sets, or the like. Additionally, the manufacturing system 262 can generate additional manufacturing data. The manufacturing system 262 can be a 3D printer. The 3D printer may be any general purpose 3D printer utilizing technologies such as Stereo-lithography (SLA), Digital Light Processing (DLP), Fused Deposition Modeling (FDM), Selective Laser Sintering (SLS), Selective laser melting (SLM), Electronic Beam Melting (EBM), Laminated object manufacturing (LOM) or the like. Other types of manufacturing devices can be used. The 3D printers can manufacture based on 3D fabrication data, which can be generated by the manufacturing system 262, system 252, or another computing device. The 3D fabrication data can include CAD data, 3D data, digital blueprints, stereolithography, or other data suitable for general purpose 3D printers. For example, the manufacturing system 262 can include a milling machine, a waterjet system, or combinations thereof, thereby providing manufacturing flexibility.

Manufacturing can be performed on site at the HCP 270 or off-site. On-site manufacturing can reduce the number of sessions with a patient and/or the time to be able to perform the surgery whereas off-site manufacturing can be useful to make the complex devices. In some embodiments, the manufacturing system 262 can be installed at the HCP 270. Off-site manufacturing facilities may have specialized manufacturing equipment. In some cases, complicated components of a surgical kit can be manufactured off-site while simpler components can be manufactured on site.

In one embodiment, information related to spinal surgeries may be displayed through a portal Graphical User Interface (GUI) of the user device 268, e.g., the smartphone device 268b discussed in connection with FIG. 17. Further, the user devices 268, 271, 272 may be any other device comprising a GUI, for example, a laptop, desktop, tablet, phablet, or other such devices known in the art. The device 268 can be used to access the patient portal (e.g., patient portal 40 of FIG. 2). The device 272 can be used to access the HCP portal (e.g., HCP portal 42 of FIG. 2) and used to approve or revise treatment protocols. The user devices 268, 271, and 272 may include client computing devices, patient computing devices, surgical devices, and physician computing devices.

The system 252 can include and/or communicate with an event detection module 286. The event detection module 286 can be trained with training items that include one or more of historical recovery data, patient surveys, healthcare provider feedback, trackable health metrics, a set of images (e.g., camera images, still images, scans, MRI scans, CT scans, X-ray images, laser scans, etc.), patient information, an implant configuration used in a successful surgery, and/or a scored surgery outcome resulting from one or more of surgeon feedback, patient recovery level, recovery time, results after a set number of years, etc. The event detection module 286 can include a patient monitoring platform that is linked to one or more patient devices 268. The event detection module 286 receives linking input from the patient authorizing the linking of the patient devices 268. Once the patient devices 268 are linked to an account of the patient, the account automatically stores the received post-operative recovery data (e.g., MRI scans, X-ray images, collected health metrics of the patient, patient feedback, etc.). In some embodiments, the patient devices 268 can communicate with one another via, for example, direct communications (e.g., Bluetooth communications, wired connections, etc.) via network communications (e.g., via a local network), or other communication means. For example, the wearable devices 268a, 268c can communicate directly with the smartphone device 268b. The smartphone device 268b can then send all or some of the collected biometric data to a remote device via the communication network 264. A user can select whether the unencrypted or encrypted data is stored and/or transmitted by the user devices. In some embodiments, the user devices can encrypt data and can transmit encrypted data that is decrypted by the system 252 or another component of the system 248.

The event detection module 286 can receive post-operative recovery data collected from the patient devices 268, analyze the post-operative recovery data, and provide an output indicating an adverse recovery event has occurred based on the inputted post-operative recovery data and a post-operative recovery plan for the patient. For example, the event detection module 286 receives post-operative recovery data (e.g., activity data of the user, drug information of the user, pain surveys, healthcare provider feedback, etc.) and determines whether the user is recovering according to a post-operative recovery plan. In a first example, the event detection module 286 determines whether the patient's activity level is above an activity threshold that can cause physical damage to or impair recovery of the patient and notifies the patient to avoid the activity level. In a second example, the event detection module 286 determines whether the patient-specific implant is causing inflammation in the patient and recommends the user reduce activity and apply an inflammation reduction object (e.g., ice, icepack, or any cold object) to reduce the inflammation. The event detection module 286 can generate one or more recommended actions based on the determinations. The event detection module 286 can perform different analyses based on the patient's current status, planned recovery, etc.

The event detection module 286 can generate a recovery timeline for a patient based on a virtual model with anatomical elements of the patient in a target configuration. The recovery timeline can include spinal metrics and predicted recovery information for the patient. For example, the timeline can illustrate to the patient how their spine will heal in the weeks, months, or years following completion of a surgical procedure. The surgical procedure can be considered complete when the instruments and sponges have been counted, dressings secured, and the surgical team has completed procedure-related activities on the patient in the surgical suite. After completion of the surgical procedure, the patient can be transported from the surgical suite to a recovery room. After recovery, the patient can be transported from the surgical suite to a hospital room or discharged.

The event detection module 286 can send the recovery timeline to a patient's device and update the timeline at any time based on a trigger, such as adjustments to the post-operative recovery plan, an adverse recovery event, and/or the post-operative recovery data. The event detection module 286 can display on the patient device pre-operative educational information, post-operative recovery assistance information, or the post-operative recovery plan. For example, the event detection module 286 provides the user with a diet and exercise routine to complete prior to the surgery, recovery predictions, recovery goals, activities to perform after surgery, a diet for the patient after the surgery, pain levels to expect, when to start physical therapy, activities to perform and avoid, etc. Pre-operatively generated recovery timelines can be sent prior to and/or after beginning the surgical procedure. Intra-operatively generated recovery timelines can be sent during and/or after the surgical procedure. Post-operatively generated recovery timelines can be sent after completion of the surgical procedure.

The system 252 can include 3D virtual models representing the anatomy of the patient to perform simulations of the patient's recovery. The system 252 can collect patient recovery data from the patient devices 268 and healthcare devices, and perform simulations with the 3D model to determine how the patient is recovering. The system 252 can convert and weight the data from the patient devices 268 using machine learning modules. For example, the system 252 can weight data from historically accurate devices more than data from historically inaccurate devices. User devices can be periodically or continuously scored and ranked for accuracy. The system 252 can compare patient recovery data to a recovery plan to determine whether the user is recovering according to the plan. Using the recovery data, the system 252 can quantify the post-operative recovery value (e.g., post-operative score and/or amount) of the patient and display the planned or predicted recovery value on a user device for the patient to view. For example, the patient can view their recovery data and their recovery progress to see if they are on track, falling behind their planned recovery, or recovering faster than planned.

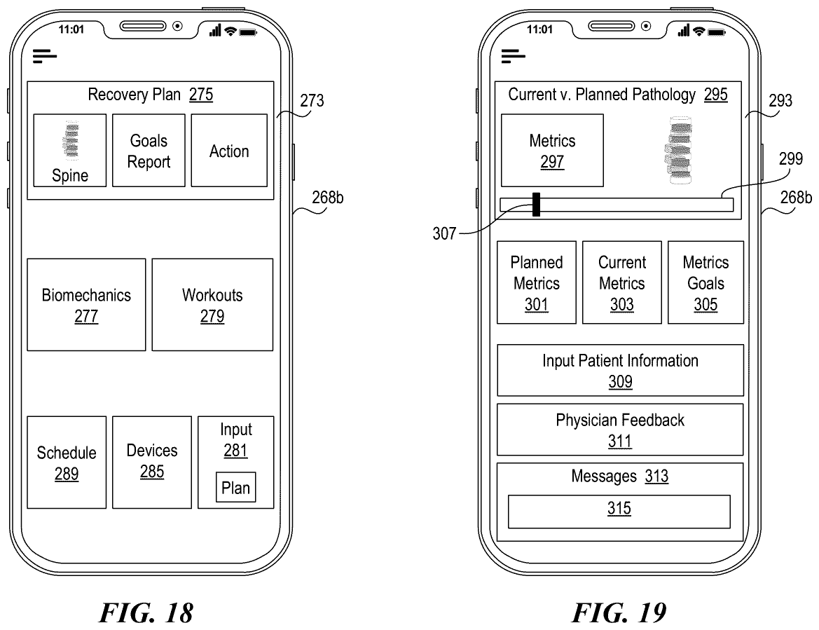

The system 252 can perform simulations based on collected patient data to generate predictive simulations (e.g., biomechanics simulations, range of motion simulations, etc.), CAD models, images, and/or 3D models for the patient to visualize how their spine will look at different time thresholds (e.g., 3 months, 6 months, 2 years, etc.) after the surgery. If recovery goals are not being met or are being exceeded, the system 252 can send notifications for the patient to provide feedback. For example, the system 252 sends a reminder for the user to attend physical therapy, suggests the user exercise regularly, requests information on the patient's pain levels, requests feedback about the recovery plan, or requests biometric data (e.g., weight, heart rate, BMI, blood sugar levels, etc.). The notifications, reminders, and other information can be displayed via the user device, as discussed in connection with FIGS. 18 and 19. In some embodiments, the system 252 generates a patient-specific monitoring plan based on data collection capabilities of the one or more linked patient devices 268 and the simulations. For example, the accuracy of the simulations can be improved based on the type of patient data (e.g., X-rays, biometric data, activity levels, diet, heart rate, sleep data, inflammation data, user feedback, etc.) that is collected from the patient devices. The post-recovery plan can be modified based on the patient-specific monitoring plan.

FIG. 18 shows a user device displaying an interactive GUI in accordance with some implementations of the disclosed technology. The user device can be the smartphone device 268b discussed in connection with FIG. 17 or other user device discussed herein. For example, the smartphone device 268b can have a screen 273 displaying the GUI for managing the post-operative recovery plan 275 and one or more buttons, such as biomechanics or tracking body movement button 277, managing workouts button 279, or inputting information button 281. The user can select the tracking body movement button 277 to start tracking movement collected by, for example, one or more wearable devices, such as wearable devices 268a, 268c of FIG. 17. In some embodiments, the tracking body movement button 277 can be selected to input biomechanics information, such as range of motion test data, workout information, biomechanics diagnostic information, test results, images of the patient's posterior, or other information related to biomechanics. A user can select the managing workouts button 279 to, for example, plan workouts, generate workouts, modify workouts, download workouts, select workouts, start workouts, end workouts, select tracking of workouts, schedule workouts, or the like. For example, a user can review the recovery plan by selecting a button within the recovery plan window 275. The user can then input workout information via the managing workouts button 279. The smartphone device 268b can notify the user when a workout should be performed, is completed, etc. A user can share workout information with, for example, healthcare providers, family members, or other authorized individuals. In some implementations, the managing workouts button 279 can be used to open a workout program that allows the user to share selected information with one or more authorized individuals. This allows for collaboration and monitoring by others. The inputting information button 281 can be used to, for example, input pain data, modify therapy schedules, manage alerts (e.g., automated alerts, scheduled alerts, or the like), manage wearable devices, store patient notes, or the like.

The screen 273 can also display a device button 285 for managing user devices associated with workouts, collecting biomechanics information, collecting biometric information, or the like. If there is a newly available device, the device button 285 can be selected to initiate a pairing process. A user can authorize pairing with the newly available device and set parameters, such as collection and sharing of data parameters. In some implementations, the devices managed using the device button 285 can be linked with a workout associated with the managing workouts button 279. Additionally or alternatively, the collected device data can be associated and shared with the biometrics program associated with the tracking body movement button 277. This allows sharing and collaboration between various locally executed programs to enhance recovery of the patient. A schedule button 289 can be used to delete, create, modify, or otherwise manage schedules. The schedules can include, without limitation, workout schedules, therapy schedules, recovery schedules, or the like. Schedule data can be shared with user devices, healthcare providers, etc.

FIG. 19 shows a user device displaying a GUI in accordance with some implementations of the disclosed technology. The user device can be the smartphone device 268b discussed in connection with FIG. 17 or other user device discussed herein. For example, the user device has a screen 293 that displays a pathology window 295 showing the pathology information (e.g., current pathology and planned pathology of a patient). In some embodiments, the pathology window 295 displays an image of the current pathology of the patient and an image of the planned post-operative anatomy of the patient for visual comparison by a patient. The pathology images can be updated to show the patient the progress of recovery. In some embodiments, including the illustrated embodiment, the anatomy of the current pathology and planned post-operative pathology can be overlaid to compare the planned and current pathologies. In some embodiments, the pathology window 295 can show time-lapse imagery of a predicted recovery and the associated pathology. This can help motivate a patient to continue with the recovery plan. For example, the pathology window 295 can show an animation of the spinal column moving towards a planned corrected pathology. The pathology window 295 can include additional information, such as metrics 297. The metrics can include, for example, the metrics discussed in connection with FIGS. 14-16. In some embodiments, the metrics window 297 can display the current metrics, predicted metrics, the difference between current and predicted metrics, and other metrics information. The planned metrics button 301, current metrics button 303, and metrics goals button 305 can be selected to control display of planned metrics, current metrics, and metrics goals, respectively. For example, the planned metrics button 301 can be used to select planned metrics. The current metrics button 303 can be used to select current metrics. The metrics goals button 305 can be used to add, modify, delete, or manage metric goals.

An input patient information button 309 can be selected to input information. The information can include, without limitation, pain levels, activity levels, health information, or the like. A physician feedback button 311 can be selected to request physician feedback. In some embodiments, the user device 272 discussed in connection with FIG. 17 can display a questionnaire for inputting information for receiving physician feedback. The physician can then answer the patient via a messages window 313. For example, SMS texts can be shown in a display window 315. Alerts, physician feedback, healthcare notifications, or other information disclosed herein can be displayed via the window 315.

In some embodiments, if the patient's activity level is above an activity threshold that can cause physical damage to the patient, the messages window 313 can, for example, notify the patient to avoid the activity level. The notification can be sent from a remote system. In another embodiment, the messages window 313 can display one or more recommendations, such as recommendations that the user reduce activity and apply an inflammation reduction object (e.g., ice, icepack, or any cold object) to reduce the inflammation.

The pathology window 295 can include one or more recovery timelines 299. The recovery timeline 299 can include, without limitation, spinal metrics (e.g., metrics displayed via the window 297 or other metrics) and predicted recovery information for the patient. For example, the recovery timeline 299 can illustrate to the patient how their spine will heal in the weeks, months, or years following a surgery. The system 248 of FIG. 17 can generate and transmit information for the recovery timeline 299. The timeline 299 can be automatically updated (e.g., continuously, periodically, in real time, etc.) based on at least one of one or more adjustments to the post-operative recovery plan, the adverse recovery event, or the post-operative recovery data. In some embodiments, a user can move a slider 307 along the recovery timeline 299. Moving the slider can cause the planned pathology at the time location corresponding to the slider position to be displayed with the current pathology. The slider 307 can be moved along the recovery timeline 299 to update the planned pathology at that point in time. For example, the slider 307 can be moved to two weeks in the future. The planned pathology in two weeks can be displayed via the pathology window 295. This allows a user to view the progress of recovery.

In some embodiments, the pathology window 295 can display one or more rates, such as fusion rates between one or more pairs of vertebrae, soft tissue recovery rates, biomechanics rates of change, or the like. The user can select the button 305 to modify metric goals based on the fusion rate. Based on the modified metric goals, the smartphone device 268b can display an updated list of current metrics associated with the modified metric goals via the button 303. When a user selects the button 303, the current metrics can include a list of metrics associated with the modified goals. This allows coordination between goals and displayed metrics and the displayed pathology.

FIG. 20 illustrates a system 352 for manufacturing implants, monitoring patients, or other processes disclosed herein. For example, the system 352 can be part of the system 252, or another system or subsystem of the system 248 of FIG. 17. In some embodiments, the system 352 can be part of a user device 268, 272 or be formed by multiple devices. In some embodiments, the system 352 can obtain implant surgery information (e.g., digital data, images of anatomy, correction procedure data, HCP data, case data, etc.), convert the implant surgery information into a form compatible with an analysis procedure, apply the analysis procedure to obtain results, and use the results to manufacture the patient-specific implant. The system 352 can be in communication with a data hub (e.g., data hub 70 of FIG. 2) and can provide a patient portal interface, manage manufacturing of the patient-specific implants, receive meta data, and other functions. Multiple systems 352 can communicate with one another via a wired or wireless connection. In some embodiments, the surgical assistance system 364 can be used to create user accounts, collect patient data, collect imaging data, and design an implant from patient data imaging data. Other I/O devices 340 can provide access to portals, inputting information to create user accounts, etc. A display 330 can display the collected patient data. Memory 350 can serve as a data hub and can store the patient data, imaging data, and other data.

The system 352 can handle the entire design and manufacturing process. In other embodiments, a physician can alter the implant configuration for further customization. An iterative design process can be employed in which the physician and system 352 work together, as discussed in connection with FIG. 1. For example, the system 352 can generate a proposed patient-specific implant. The physician can identify characteristics of the implant to be changed and can input potential design changes. For example, the physician can use a computing device (e.g., device 272 of FIG. 17) to view the implant design and then provide input for changing the design. The system 352 can analyze the feedback from the physician to determine a refined patient-specific implant design and to produce a patient-specific model. This process can be repeated any number of times until arriving at a suitable design. Once approved, the implant can be manufactured based on the selected design.

The system 352 can include a surgical assistance system 364. In some embodiments, the surgical assistance system 364 can include one or more protocol modules, design modules, or other modules discussed in connection with FIG. 17. The surgical assistance system 364 can analyze implant surgery information, for example, into arrays of integers or histograms, segment images of anatomy, manipulate relationships between anatomical elements, convert patient information into feature vectors, or extract values from the pre-operative plan. The system 352 can store implant surgery information analyzed by the surgical assistance system 364. The stored information can include received images of a target area, such as MRI scans of a spine, digital images, X-rays, patient information (e.g., sex, weight, etc.), virtual models of the target area, a database of technology models (e.g., CAD models), and/or a surgeon's pre-operative plan.

In some implementations, surgical assistance system 364 can analyze patient data to identify or develop a corrective procedure, identify anatomical features, etc. The anatomical features can include, without limitation, vertebra, vertebral discs, bony structures, or the like. The surgical assistance system 364 can determine the implant configuration based upon, for example, a corrective virtual model of the subject's spine, risk factors, surgical information (e.g., delivery paths, delivery instruments, etc.), or combinations thereof. In some implementations, the physician can provide the risk factors before or during the procedure. Patient information can include, without limitation, patient sex, age, bone density, health rating, or the like.

In some implementations, the surgical assistance system 364 can apply analysis procedures by supplying implant surgery information to a machine learning model trained to select implant configurations. For example, a neural network model can be trained to select implant configurations for a spinal surgery. The neural network can be trained with training items each comprising a set of images (e.g., camera images, still images, scans, MRI scans, CT scans, X-ray images, laser scans, etc.) and patient information, an implant configuration used in the surgery, and/or a scored surgery outcome resulting from one or more of: surgeon feedback, patient recovery level, recovery time, results after a set number of years, etc. This neural network can receive the converted surgery information and provide output indicating the implant configuration.

The surgical assistance system 364 can generate one or more virtual models (e.g., 2D models, 3D models, CAD models, etc.) for designing and manufacturing items. For example, the surgical assistance system 364 can build a virtual model of a surgery target area suitable for manufacturing surgical items, including implants. The surgical assistance system 364 can also generate implant manufacturing information, or data for generating manufacturing information, based on the computed implant configuration. The models can represent the patient's anatomy, implants, candidate implants, etc. The model can be used to (1) evaluate locations (e.g., map a negative 2D or 3D space), (2) select a bounding anatomical feature, such as a vertebral endplate, (3) create a best-fit virtual implant, (4) define a perimeter of the anatomical feature, and/or (5) extrude a volume defined by the perimeter and perpendicular to, for example, a best-fit plane to the interface of another anatomical feature. Anatomical features in the model can be manipulated according to a corrective procedure. Implants, instruments, and surgical plans can be developed based on the pre- or post-manipulated model. Neural networks can be trained to generate and/or modify models, as well as other data, including manufacturing information (e.g., data, algorithms, etc.).

In another example, the surgical assistance system 364 can apply the analysis procedure by performing a finite element analysis on a generated three-dimensional model to assess, for example, stresses, strains, deformation characteristics (e.g., load deformation characteristics), fracture characteristics (e.g., fracture toughness), fatigue life, etc. The surgical assistance system 364 can generate a three-dimensional mesh to analyze the model. Machine learning techniques to create an optimized mesh based on a dataset of vertebrae, bones, implants, tissue sites, or other devices. After performing the analysis, the results could be used to refine the selection of implants, implant components, implant type, implantation site, etc.

The surgical assistance system 364 can perform a finite element analysis on a generated three-dimensional model (e.g., models of the spine, vertebrae, implants, etc.) to assess stresses, strains, deformation characteristics (e.g., load deformation characteristics), fracture characteristics (e.g., fracture toughness), fatigue life, etc. The surgical assistance system 364 can generate a three-dimensional mesh to analyze the model of the implant. Based on these results, the configuration of the implant can be varied based on one or more design criteria (e.g., maximum allowable stresses, fatigue life, etc.). Multiple models can be produced and analyzed to compare different types of implants, which can aid in the selection of a particular implant configuration.

The surgical assistance system 364 can incorporate results from the analysis procedure in suggestions. For example, the results can be used to suggest a treatment protocol or plan (e.g., a PLIF plan, a TLIF plan, a LLIF plan, a ALIF plan, etc.), select and configure an implant for a procedure, annotate an image with suggested insertions points and angles, generate a virtual reality or augmented reality representation (including the suggested implant configurations), provide warnings or other feedback to surgeons during a procedure, automatically order the necessary implants, generate surgical technique information (e.g., insertion forces/torques, imaging techniques, delivery instrument information, or the like), etc. The suggestions can be specific to implants. In some procedures, the surgical assistance system 364 can also be configured to provide suggestions for conventional implants. In other procedures, the surgical assistance system 364 can be programmed to provide suggestions for patient-specific or customized implants. The suggestion for the conventional implants may be significantly different from suggestions for patient-specific or customized implants.

The system 352 can simulate procedures using a virtual reality system or modeling system. One or more design parameters (e.g., dimensions, implant configuration, instrument, guides, etc.) can be adjusted based, at least in part, on the simulation. Further simulations (e.g., simulations of different corrective procedures) can be performed for further refining implants. In some embodiments, design changes are made interactively with the simulation and the simulated behavior of the device based on those changes. The design changes can include material properties, dimensions, or the like.

The surgical assistance system 364 can improve efficiency, precision, and/or efficacy of implant surgeries by providing more optimal implant configuration, surgical guidance, customized surgical kits (e.g., on-demand kits), etc. This can reduce operational risks and costs produced by surgical complications, reduce the resources required for preoperative planning efforts, and reduce the need for extensive implant variety to be prepared prior to an implant surgery. The surgical assistance system 364 provides increased precision and efficiency for patients and surgeons.

In orthopedic surgeries, the surgical assistance system 364 can select or recommend implants, surgical techniques, patient treatment plans, or the like. In spinal surgeries, the surgical assistance system 364 can select interbody implants, pedicle screws, and/or surgical techniques to make surgeons more efficient and precise, as compared to existing surgical kits and procedures. The surgical assistance system 364 can also improve surgical robotics/navigation systems, and provide improved intelligence for selecting implant application parameters. For example, the surgical assistance system 364 empowers surgical robots and navigation systems for spinal surgeries to increase procedure efficiency and reduce surgery duration by providing information on types and sizes, along with expected insertion angles. In addition, hospitals benefit from reduced surgery durations and reduced costs of purchasing, shipping, and storing alternative implant options. Medical imaging and viewing technologies can integrate with the surgical assistance system 364, thereby providing more intelligent and intuitive results. The healthcare provider can provide feedback about the output from the surgical assistance system 364. The surgical assistance system 364 can use the feedback to adjust treatment protocols, implant designs (e.g., configurations, materials, etc.), etc.

The system 352 can include one or more input devices 320 that provide input to the processor(s) 345 (e.g., CPU(s), GPU(s), HPU(s), etc.), notifying it of actions. The input devices 320 can be used to manipulate a model of the spine. The actions can be mediated by a hardware controller that interprets the signals received from the input device and communicates the information to the processors 345 using a communication protocol. Input devices 320 include, for example, a mouse, a keyboard, a touchscreen, an infrared sensor, a touchpad, a wearable input device, a camera- or image-based input device, a microphone, or other input devices (e.g., devices 268 and 272 of FIG. 17). Processors 345 can be a single processing unit or multiple processing units in a device or distributed across multiple devices. Processors 345 can be coupled to other hardware devices, for example, with the use of a bus, such as a PCI bus or SCSI bus.

The system 352 can include a display 330 used to display text, models, virtual procedures, surgical plans, implants, and graphics. In some implementations, the display 330 can display the patient portal, HCP portal, or other portals or interfaces. The image(s) or models can be displayed and manipulated using typical touch-screen gestures via the display 330 (e.g., when the display 330 is part of the devices 268 and/or 272 of FIG. 17), including zoom, pan, and rotate. In other embodiments, other I/O devices 340 are used to zoom, pan, rotate, or otherwise manipulate the images. In some implementations, the display 330 provides graphical and textual visual feedback to a user. In some implementations, the display 330 includes the input device as part of the display, such as when the input device is a touchscreen or is equipped with an eye direction monitoring system. The processors 345 can communicate with a hardware controller for devices, such as for a display 330. In some implementations, the display is separate from the input device. Examples of display devices include an LCD display screen, an LED display screen, a projected, holographic, or augmented reality display (such as a heads-up display device or a head-mounted device), and so on. Other I/O devices 340 can also be coupled to the processors 345, such as a network card, video card, audio card, USB, firewire or other external device, camera, printer, speakers, CD-ROM drive, DVD drive, disk drive, or Blu-Ray device. Other I/O devices 340 can also include input ports for information from directly connected medical equipment such as imaging apparatuses, including MRI machines, X-ray machines, CT machines, etc. Other I/O devices 340 can further include input ports for receiving data from these types of machines from other sources, such as across a network or from previously captured data, for example, stored in a database.

In some implementations, the system 352 also includes a communication device capable of communicating wirelessly or wire-based with a network node. The communication device can communicate with another device or a server through a network using, for example, TCP/IP protocols. System 352 can utilize the communication device to distribute operations across multiple network devices, including imaging equipment, manufacturing equipment, etc.

The system 352 can include memory 350. The processors 345 can have access to the memory 350, which can be in a device or distributed across multiple devices. Memory 350 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. Memory 350 can include program memory 360 that stores programs and software, such as an operating system 362, surgical assistance system 364, and other application programs 366, such as program for managing a data hub. Memory 350 can also include data memory 370 that can include, e.g., patient data (name, DOB, gender, contact information, consent, scan data, etc.), implant information, configuration data, settings, user options or preferences, etc., which can be provided to the program memory 360 or any element of the system 352, such as a manufacturing system 367. The description of the manufacturing system 262 of FIG. 17 applies equally to the manufacturing system 367 of FIG. 20.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTER VERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 18/384,762, filed Oct. 28, 2023, titled “SYSTEMS AND METHODS FOR SELECTING, REVIEWING, MODIFYING, AND/OR APPROVING SURGICAL PLANS;”
U.S. application Ser. No. 18/537,600, filed Dec. 12, 2023, titled “PATIENT-SPECIFIC IMPLANT DESIGN AND MANUFACTURING SYSTEM WITH A REGULATORY AND REIMBURSEMENT MANAGER;”
International Application No. PCT/US24/10202, filed Jan. 3, 2024, titled “PATIENT-SPECIFIC SPINAL FUSION DEVICES AND ASSOCIATED SYSTEMS AND METHODS;”
U.S. application Ser. No. 18/408,409, filed Jan. 9, 2024, titled “SYSTEM FOR EDGE CASE PATHOLOGY IDENTIFICATION AND IMPLANT MANUFACTURING;”
U.S. application Ser. No. 18/408,452, filed Jan. 9, 2024, titled “SYSTEM FOR MODELING PATIENT SPINAL CHANGES;”
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER;”
U.S. Application No. 63/539,797, filed Sep. 21, 2023, titled “ROTATABLE INTERVERTEBRAL IMPLANTS FOR TRANSFORAMINAL LUMBAR INTERBODY FUSION TECHNIQUES;” and
U.S. Application No. 63/542,264, filed Oct. 3, 2023, titled “PATIENT-SPECIFIC SURGICAL POSITIONING GUIDES AND METHODS OF MAKING AND USING THE SAME.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

While embodiments have been shown and described, various modifications may be made without departing from the scope of the inventive concepts disclosed herein.

1. A computer-implemented method, comprising:
training an event detection module based on historical recovery data training items;
post-operatively monitoring a patient that has undergone a surgical procedure by:
linking one or more patient devices to a patient monitoring platform,
receiving, at the patient monitoring platform, post-operative recovery data of the patient collected by the one or more linked patient devices,
inputting the post-operative recovery data into the trained event detection module, and
determining, using the trained event detection module, an adverse recovery event has occurred based on the inputted post-operative recovery data and a post-operative recovery plan for the patient; and
performing, via the patient monitoring platform, a post-operative recovery action based on the adverse recovery event to assist the patient with recovery from the surgical procedure.

2. The computer-implemented method of claim 1, further comprising:
obtaining a virtual model representing an anatomy of the patient;
generating a recovery timeline for the patient based on the virtual model with anatomical elements of the patient in a target configuration, wherein the recovery timeline includes one or more spinal metrics and predicted recovery information for the patient; and
transmitting the recovery timeline for review by a user, wherein the recovery timeline is automatically updated based on at least one of:
one or more adjustments to the post-operative recovery plan,
the adverse recovery event, or
the post-operative recovery data.

3. The computer-implemented method of claim 1, further comprising:
determining, using the patient monitoring platform, the post-operative recovery action for the patient based on the adverse recovery event; and
monitoring the post-operative recovery action using at least one of the one or more linked patient devices.

4. The computer-implemented method of claim 1, further comprising:
displaying, via a patient portal for the patient, at least one of pre-operative educational information, post-operative recovery assistance information, or the post-operative recovery plan.

5. The computer-implemented method of claim 1, further comprising causing a graphical user interface to be displayed on a user device for managing at least one of uploading of image data, collecting of patient information, or setting goals.

6. The computer-implemented method of claim 1, further comprising dynamically modifying the post-operative recovery plan based on newly available patient data and a simulation using a virtual model representing an anatomy of the patient.

7. The computer-implemented method of claim 1, further comprising:
generating a three-dimensional virtual model representing an anatomy of the patient according to newly available patient data; and
generating a new post-operative recovery plan based on the three-dimensional virtual model.

8. The computer-implemented method of claim 1, further comprising:
receiving patient data associated with the post-operative recovery action;
quantifying a recovery amount associated with the post-operative recovery based on the received patient data; and
causing display, via the one or more linked patient devices, of the recovery amount for viewing by the patient.

9. The computer-implemented method of claim 1, wherein the post-operative recovery plan includes a recovery timeline for the patient, wherein the recovery timeline includes one or more recovery data thresholds selected for comparison to the post-operative recovery data.

10. The computer-implemented method of claim 1, further comprising:
performing one or more simulations of the patient recovering from the surgical procedure;
generating a patient-specific monitoring plan based on data collection capabilities of the one or more linked patient devices and the one or more simulations; and
generating the post-operative recovery plan based on the patient-specific monitoring plan.

11. The computer-implemented method of claim 10, further comprising:
obtaining newly available patient image data of the patient after completion of a portion of the post-operative recovery plan;
determining a candidate modification to at least one of the post-operative recovery plan or the patient-specific monitoring plan based on the newly available patient image data;
simulating an effect on the patient based on the candidate modification; and
in response to the effect exceeding a threshold, modifying the post-operative recovery plan according to the candidate modification.

12. The computer-implemented method of claim 1, further comprising:
receiving linking input from the patient authorizing the linking of the one or more patient devices; and
linking the one or more patient devices to an account of the patient based on the linking input, wherein the account automatically stores the received post-operative recovery data.

13. The computer-implemented method of claim 1, wherein the post-operative recovery plan includes target post-operative recovery data, the method further comprising:
comparing the collected post-operative recovery data to the target post-operative recovery data; and
in response to the collected post-operative recovery data deviating from the target post-operative recovery data by a value above, below, or at a threshold, determining the post-operative recovery action.

14. The computer-implemented method of claim 1, wherein the post-operative recovery action is at least one of:
an alert for at least one of a physician or a selected individual associated with the patient,
a goal reminder viewable by the patient for the post-operative recovery plan,
a feedback request for the patient, or
an activity request for the patient.

15. The computer-implemented method of claim 1, wherein the post-operative recovery data includes one or more of biometric data, questionnaire data, or exercise data.

16. A system comprising:
one or more processors; and
one or more memories storing instructions that, when executed by the one or more processors, cause the system to perform a process comprising:
training an event detection module based on historical recovery data training items;
post-operatively monitoring a patient that has undergone a surgical procedure by:
linking one or more patient devices to a patient monitoring platform,
receiving, at the patient monitoring platform, post-operative recovery data of the patient collected by the one or more linked patient devices,
inputting the post-operative recovery data into the trained event detection module, and
determining, using the trained event detection module, an adverse recovery event has occurred based on the inputted post-operative recovery data and a post-operative recovery plan for the patient; and
performing, via the patient monitoring platform, a post-operative recovery action based on the adverse recovery event to assist the patient with recovery from the surgical procedure.

17. The system of claim 16, wherein the process further comprises:
obtaining a virtual model representing an anatomy of the patient;
generating a recovery timeline for the patient based on the virtual model with anatomical elements of the patient in a target configuration, wherein the recovery timeline includes one or more spinal metrics and predicted recovery information for the patient; and
transmitting the recovery timeline for review by a user, wherein the recovery timeline is automatically updated based on at least one of:
one or more adjustments to the post-operative recovery plan,
the adverse recovery event, or
the post-operative recovery data.

18. The system of claim 16, wherein the process further comprises:
determining, using the patient monitoring platform, the post-operative recovery action for the patient based on the adverse recovery event; and
monitoring the post-operative recovery action using at least one of the one or more linked patient devices.

19. The system of claim 16, wherein the process further comprises:
displaying, via a patient portal for the patient, at least one of pre-operative educational information, post-operative recovery assistance information, or the post-operative recovery plan.

20. The system of claim 16, wherein the process further comprises:
causing a graphical user interface to be displayed on a user device for managing at least one of uploading of image data, collecting of patient information, or setting goals.

21. A non-transitory computer-readable medium storing instructions that, when executed by a computing system, cause the computing system to perform operations comprising:
training an event detection module based on historical recovery data training items;
post-operatively monitoring a patient that has undergone a surgical procedure by:
linking one or more patient devices to a patient monitoring platform,
receiving, at the patient monitoring platform, post-operative recovery data of the patient collected by the one or more linked patient devices,
inputting the post-operative recovery data into the trained event detection module, and
determining, using the trained event detection module, an adverse recovery event has occurred based on the inputted post-operative recovery data and a post-operative recovery plan for the patient; and
performing, via the patient monitoring platform, a post-operative recovery action based on the adverse recovery event to assist the patient with recovery from the surgical procedure.

22. The non-transitory computer-readable medium of claim 21, wherein the operations further comprise:
obtaining a virtual model representing an anatomy of the patient;
generating a recovery timeline for the patient based on the virtual model with anatomical elements of the patient in a target configuration, wherein the recovery timeline includes one or more spinal metrics and predicted recovery information for the patient; and
transmitting the recovery timeline for review by a user, wherein the recovery timeline is automatically updated based on at least one of:
one or more adjustments to the post-operative recovery plan,
the adverse recovery event, or
the post-operative recovery data.

23. The non-transitory computer-readable medium of claim 21, wherein the operations further comprise:
determining, using the patient monitoring platform, the post-operative recovery action for the patient based on the adverse recovery event; and
monitoring the post-operative recovery action using at least one of the one or more linked patient devices.

24. The non-transitory computer-readable medium of claim 21, wherein the operations further comprise:
displaying, via a patient portal for the patient, at least one of pre-operative educational information, post-operative recovery assistance information, or the post-operative recovery plan.

25. The non-transitory computer-readable medium of claim 21, wherein the operations further comprise:
causing a graphical user interface (GUI) to be displayed on a user device for managing at least one of uploading of image data, collecting of patient information, or setting goals.

26-41. (canceled)
